(19) United States
(12) Patent Application Publication
(10) Pub. No.: US 2024/0379187 A1
(43) Pub. Date: Nov. 14, 2024

(54) SYSTEMS AND METHODS FOR OMICS-BASED ANALYSIS OF GENE EXPRESSION

(71) Applicant: Arizona Board of Regents on Behalf of the University of Arizona, Tucson, AZ (US)

(72) Inventors: Gayatri Vedantam, Tucson, AZ (US); V.K. Viswanathan, Tucson, AZ (US); Bryan Roxas, Tucson, AZ (US)

(21) Appl. No.: 18/662,558

(22) Filed: May 13, 2024

Related U.S. Application Data
(60) Provisional application No. 63/501,620, filed on May 11, 2023.

(51) Int. Cl. G16B 25/00; C12Q 1/689; C12Q 1/6895

(52) U.S. Cl. CPC G16B 25/00; C12Q 1/689; C12Q 1/6895; C12Q 2600/158

(57) ABSTRACT

Infectious diseases pose persistent threats to the health and wellbeing of humans and animals globally. Systems and methods for multi omics-based validation of gene expression data have been developed. The methods assess disease/infection/immunity data and identify treatment regimens most likely to yield beneficial patient outcomes. The methods are implemented in a computational program for integrated, queryable pathogen/host atlas of CDC “Urgent Threat” Pathogens. Methods of treatment for disease/infection/immunity using active agents according to the described methods are also provided. In some forms, the systems and methods determine optimal treatment regimens for subjects infected with Clostridioides difficile, or Neisseria gonorrhoeae. Exemplary subjects include farm animals and humans.

### CROSS-REFERENCE TO RELATED APPLICATIONS

This application claims the benefit of and priority to U.S. Provisional Application No. 63/501,620 filed May 11, 2023, which is hereby incorporated by reference in its entirety.

### STATEMENT REGARDING FEDERALLY SPONSORED RESEARCH

This invention was made with government support under Grant No. AI169791 awarded by National Institute of Health. The government has certain rights in the invention.

### FIELD OF THE INVENTION

The disclosed invention is generally in the field of disease assessment and treatment and more specifically in using gene expression analysis associated with microbial responses to antimicrobial treatment to assess and treat infectious diseases.

### BACKGROUND OF THE INVENTION

Infectious diseases pose a persistent threat to the health and wellbeing of humans and animals around the world. Climate change and large-scale geopolitical perturbations (e.g., migrations) uncover infection landscapes, and expose populations to pathogens (animal and human). At no time has this been more apparent than in the past 2 years of SARS-Cov-2 emergence and global dissemination.

C. difficile is considered an Urgent Threat to US healthcare by the CDC (Finn, et al., BMC Infectious Diseases;21 (1): 456 (2021)). Annual cases range from 500,000-1,000,000 resulting in ˜29,000 deaths and >$6.3 billion cost overall to healthcare systems. There are currently no vaccines against C. difficile infections, and there are several limitations to antibiotic therapy, including the emergence of resistant strains. The mechanisms by which C. difficile causes disease are poorly understood and are an active area of investigation. The ˜4,000 genes of C. difficile are controlled by complex regulatory networks that are responsive to metabolic and environmental cues; the functions of most of these genes in bacterial physiology and virulence remain undefined. Understanding their roles in pathogenesis could facilitate the development of therapeutic agents. There are extensive publicly-available datasets of bacterial pathogen gene expression under different conditions; these include transcriptomic (RNAseq and Microarray) and proteomics data, as well as regulatory and structural information. Sources include the Gene Expression Omnibus and ProteomeExchange, and publications (typically as supplemental data). In the infectious disease research context, information is invariably generated for diverse clinical isolates and respective mutant derivatives. Unfortunately, these data are not readily accessible or queryable.

Therefore, it is an object of the invention to provide enhanced methods of providing centralized access to disparate datasets, and embedded tools for comparative analysis.

It is another object of the invention to provide systems and methods for rapid and effective analysis and reporting of organismal, e.g., microbial, physiology and pathogenesis.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### SUMMARY OF THE INVENTION

Methods for combining and reorganizing genomic and transcriptomic data for an organism of interest in the form of a searchable and interactive database have been developed. In some forms, the organism is a microorganism, such as a pathogenic microorganism. The database enables the analysis of homology and/or differential expression of one or more user-defined genes and/or gene functions from a pool of genomic sequence data derived from a multiplicity of samples of the organism, e.g., pathogenic microorganisms of the same species, are provided. In some forms, the methods include determining homology between the multiplicity of samples to identify common genes; identifying differential expression of the common genes; identifying virulence factors, and/or identifying or characterizing host-pathogen interactions; and presenting data reporting the homology and/or differential expression of the user-defined genes and/or gene functions. In some forms, the presenting includes identifying relationships between one or more genes within samples of the multiplicity of samples. Typically, the methods are implemented on a computer, and the pool of genomic sequence data is provided in the form of a computer-readable database. In some forms, the computer-readable database includes one or more of gene expression data, transcriptomics data, protein abundance data, proteomics data. Exemplary databases include the Gene Expression Omnibus (GEO) database and/or the Proteome Xchange database, the Gene Ontology database, KEGG database, RAST Subsystems database, and Enzyme Classes.

In some forms the methods include one or more of: (a) determining sequence homology between two or more of the multiplicity of samples of an organism, e.g., microorganism; (b) determining differential gene expression and/or abundance, including log2 ratio, median normalization and one-sample t-test between two or more of the multiplicity of samples of the organism; (c) principal component analysis; and (d) data structuring, including column identity and position for data points. In some forms the methods include the creating, storing, updating and/or retrieving of data including quantitative proteomic analysis and/or transcriptomic analysis two or more as a relational database using structured query language (SQL). In some forms, the methods present data implemented through one or more computer programs including RShiny, RBioconductor, RStudio Connect, gene set enrichment analysis (GSEA), and/or GEO2R. Typically, the methods present results as visualization of complex data, such as a heat map representation of changes in the expression of one or more genes among two or more of the multiplicity of samples.

Typically, the analysis of an SQL database is initiated by one or more user-defined input terms entered through a user interface connected with the computer. Exemplary user-defined input is selected from the name of a gene, a gene function, a gene mutation, a nucleic acid sequence, the name of an organelle, a genetic pathway, a sub-species or clade, the name of a polypeptide, an amino acid sequence, a gene expression pathway, the name of a toxin, the name of a disease or disorder, the name of a virus, the name of a geographic location or place, a date, a range of dates, the name of a drug, and a host cell or organism, the name of an investigator or scientific institution, and the name of a methodology; or combinations thereof. In an exemplary form, the user-defined input is the name of a gene or a code corresponding to a gene, and the presenting includes identifying one or more polymorphisms within the gene in the pool of genomic sequence data. In further forms, the presenting also identifies the sample(s) associated with each polymorphism. In some forms, the user-defined input term is the name of a gene or a code corresponding to a gene, and the methods present results including differential expression of the gene amongst the pool of genomic sequence data. In some forms, the user-defined input parameters include selecting a gene in one or more from the group including pangenome, core genome, metabolic core genome, and essential genes; and/or selecting a gene from the cell localization cytoplasm or from cytoplasmic membrane; and/or selecting an experiment parameter filter from the group including response to specific gene knockout, bile acid, antibacterial, antibiotic, and stress.

Typically, the methods provide greater-depth of understanding and/or provide insight to the pool of data for a pathogenic microorganism associated with one or more diseases or disorders in humans. In some forms, the methods determine or correlate expression of one or more genes expressed by the pathogenic microorganism that is known to be associated with resistance to one or more antimicrobial agents, such that a high gene expression compared to a median gene expression informs the user that the microorganism has an increased chance of survival in the presence of the one or more antimicrobial agents.

In some forms, the methods include correlating treatment options for an infection of a subject with the pathogenic microorganism(s) with gene expression to determine clinical outcome, whereby a high gene expression compared to a median gene expression signature score indicates that the subject has a lower chance of a positive clinical outcome when treated with one or more therapeutic agents that are associated with the one or more genes expressed by the microorganism. In some forms, the positive clinical outcome is therapeutic efficacy of the antimicrobial agent, and/or survival of the subject.

In some forms, the methods calculate changes in gene expression between two or more samples/data points as determined by a log2 transformation.

In some forms, a median or reference value is determined by expression of genes of a reference strain of the organism. In some forms, the methods include treating a subject in need thereof for an infection or disease or disorder with the organism (e.g., pathogenic microorganism, cancer cells, etc.) by administering to the subject the active agent, e.g., antimicrobial or other therapeutic agent in an amount effective to treat the infection or disease or disorder if the organism (e.g., pathogenic microorganism, cancer, etc.) has a gene expression score for one or more genes that is equal to or less than a control value.

Methods for identifying molecular pathways in one or more organism, e.g., pathogenic microbial organism(s), e.g., bacterial strain(s), in response to an active agent are also provided. The methods typically include (a) contacting a microbe (e.g., bacterium) of a first microbial (e.g., bacterial) strain with a first active agent; (b) determining a gene expression score for one or more genes of the first microbial (e.g., bacterial strain) in the presence of the active agent, whereby the determining optionally further includes evaluating gene expression and dose response data for the first active agent; and (c) analyzing genes that demonstrate expression and dose response correlations to identify significantly represented molecular pathways, wherein a gene expression score greater or lower than a control value identify genes in the first microbial (e.g., bacterial) strain(s) responsive to the first active agent. Typically, the control value is the gene expression score of a wild type strain of the microbial (e.g., bacterial) strain in the absence of the active agent. In some forms the methods further include (d) determining differences in gene expression between the first and a second or further microbial (e.g., bacterial) strain in the presence of the same active agent, whereby the differences identify molecular pathways that are responsible for different phenotypic and/or genotypic responses to the first active agent. In some forms the methods further include (e) compiling the gene expression data for the first and second or further microbial (e.g., bacterial) strains in the presence of the first active agent in a database, whereby the data base is searchable. In some forms the database further includes gene expression data for a multiplicity of different microbial (e.g., bacterial) strains, and/or a multiplicity of different active agents. In some forms the methods further include wherein the first active agent is an antimicrobial agent, and wherein the phenotypic and/or genotypic responses to the first active agent include susceptibility or resistance to the antimicrobial agent. In some forms the methods further include treating a subject having an infection caused by a pathogenic microorganism with the first active agent when the pathogenic microorganism includes molecular pathways that are associated with susceptibility to the first active agent, and whereby the molecular pathways of the pathogenic microorganism are determined by comparing gene expression data of the pathogenic microorganism to those in the database. Alternatively, in other forms, the methods include not treating a subject with the first active agent when the subject has an infection caused by a pathogenic microorganism including molecular pathways that are responsible for resistance to the first antimicrobial agent, whereby the molecular pathways of the pathogenic microorganism are determined by comparing gene expression data of the pathogenic microorganism to those in the database.

In some forms, the pathogenic microorganism is selected from the group including Clostridioides spp., Neisseria spp., Candida spp., Enterobacteriaceae spp., Acinetobacter spp., Campylobacter spp., and Escherichia spp. For example, in some forms, the pathogenic microorganism is selected from the group including Clostridioides difficile, Neisseria gonorrhoeae, Candida auris, and Escherichia coli. In certain forms, the pathogenic microorganism is a strain of Clostridioides difficile. In other forms, the antibiotic-resistant bacterium is a carbapenem-resistant Enterobacteriales sp. or a carbapenem-resistant Acinetobacter sp.

Related methods are also provided for other organisms including, but not limited to, cancer cells, etc.

### BRIEF DESCRIPTION OF THE DRAWINGS

The accompanying drawings, which are incorporated in and constitute a part of this specification, illustrate several forms of the disclosed method and compositions and together with the description, explain the principles of the disclosed method and compositions.

FIG. 1 is a schematic representation of four components combined within the concept of multi-omics database methodologies, including (i) a multiplicity of samples of multiple clinical isolates, (ii) from multiple laboratories, (iii) multiple types of data including proteomic, transcriptomic and genomic datasets, and (iv) multiple sources, including published and unpublished data as well as publicly available databases.

FIG. 2 is a diagram showing exemplary results of an analysis using the input term “spore”, with results of three different search systems. The number of genes identified by searching each of three databases is indicated as a Venn diagram displaying numbers of genes identified in each search (181, 31, 48, 8 and 2 in each sector, respectively.

FIGS. 3A-3B are schematic representations of the workflow of the Centralized Access to Gene Expression Datasets (CAT-GxD) system, showing three stages of curation and integration of data from distinct databases/sources to form an integrated database; input of various user-defined search terms, cut-off values, etc., to form a customized data frame (FIG. 3A); and output of results tailored to the analysis requested by the user in the form of an interactive dashboard (FIG. 3B). The exemplified interactive dashboard includes a Heatmap tab (visual representation of the expression changes for the selected genes or groups of genes under the specified conditions), a PCA (Principal Component Analysis) tab for clustering of data for each condition, study information table, a Gene Info Table tab (for interactions, if any, between the gene products as defined by String), STRING network analysis and KEGG pathway maps as well as hyperlinks for accessing database entries and any related publication(s), from which the data were derived.

FIG. 4 is a schematic representation of the workflow for the computational processes and data sources implemented within CAT-GxD, including a database of publicly available data that undergoes “Data wrangling”, including ID Matching, Differential Expression, and Data structuring to form a SQL database that is then interrogated by user-defined search terms to provide results in the form of an interactive dashboard that is rendered from the SQL database.

FIGS. 5A-5E are images of the interactive dashboard implemented in CAT-GxD, which provides a visual display of data resulting from the user-defined search of an SQL database, depicted as viewed by the user when displayed on a screen. FIG. 5A shows the left side of the dashboard, including a user-interface for input of terms and values used to reorganize the data and to change threshold values for differences in expression, etc. Search/selection input fields include omics-type, category, study, ratio, gene filters, gene keyword search, location, gene set, enzyme class, cog, RAST subsystems, GO biological process, GO molecular function, UniprotKB keywords, and selection of [log2] threshold, heatmap output color selection, option for hierarchical clustering and data download choices; these filters determine the list of genes, shown on the right side of the screen (depicted in FIGS. 5B-5E). FIG. 5B shows the dashboard options under the default Gene Info tab, for which expression data will be compared. The table lists description of the gene and its location within an operon. Clicking on the gene name will open the AlphaFold structure page for the corresponding protein. Also listed are the isoelectric point (pi), molecular weight (mw), length and predicted location of the corresponding protein. FIG. 5C shows the display of results for the search term/biology (e.g., “superoxide”) as a heatmap, with experiments depicted on the X-axis and genes depicted on the Y-axis. Clustering shows similar changes under different experimental conditions and the display allows the user to hover over any portion of the gene map to see the reported log2 ratio, the gene name, and the study. FIG. 5D shows the information under the second tab “Study Info”. This lists all the omics datasets compiled under CAT-GxD. The study details and corresponding publications, if any, can be accessed by clicking on the names in columns 3 and 4, respectively. FIG. 5E is an image of the display of results under the “Network” tab, depicting interactions between the gene products, with each gene depicted as a circle including a gene ID number, with lines joining circles depicting connections between genes. Also shown are input tabs for each of the parameters STRING score and ratio.

FIG. 6 is an image of a heatmap generated by the Use case exemplified in the Examples, showing 28 downregulated genes that were inputted into the search bar to generate a heatmap. 123 of the 144 ratios had gene expression or protein abundance ratios greater or less than the log2 ratio cutoff of +1 or −1. 20 ratios, including the reference ratio of sig54, were selected in ‘Data Frame’ tab to reduce the size and complexity of the heatmap, highlighting ratios that have similar and dissimilar expression patterns to sig54.

FIG. 7 is an image of a graph depicting C. difficile Principal Component Analysis (PCA) generated by the Use case exemplified in the Examples, showing 3 ratios, the reference (sig54), the most similar (240 μM DCA 48 hrs) and the most dissimilar (80 μM succinate 24 hrs biofilm) that were selected for more detailed analysis using CAT-GxD's PCA, network and pathway analysis features. Both DCA and succinate induced biofilm formation in C. difficile with the latter inducing a significantly thicker biofilm. 3 gene clusters were observed from principal component analysis (PCA) of the 3 ratios: [1] an anaerobic glutamate/electron transfer flavoprotein (hadA . . . 5 . . . ctfA1 . . . [467033 . . . 474494]) cluster, [2] a phosphotransferase system (PTS) (CD0284 . . . 5 . . . CD0290 . . . [346714 . . . 350720]) and sporulation membrane/peptidases (CD2697 . . . 1 . . . CD2699 [3117803 . . . 3118351]) cluster, and [3] a butanoate metabolism (buk . . . 2 . . . CD2382 [2745688 . . . 2748166]) and pentose/glucuronate interconversion (CD2323. . . . CD2324 . . . [2686241 . . . 2686241]) and uronic acid metabolism (kdgT1 . . . 1 . . . uxaA . . . [3357631 . . . 3360077]). These 3 clusters represented genes/operons with similar expression patterns based on the 3 selected ratios.

FIG. 8 is an image of a graph depicting C. difficile Search Tool for the Retrieval of Interacting Genes/Proteins (STRING) Network Analysis generated by the Use case exemplified in the Examples, that shows CAT-GxD's network analysis features which allows the 28 downregulated genes to be viewed as a network defined by interactions from the STRING database. Two genes without any interactions within the 28 inputted genes, CD1413 and CD3093, are not included in the network. In order to link the separate networks, the 26 genes were sent to the STRING database by clicking on the STRING icon.

FIG. 9 is an image of a graph depicting C. difficile Kyoto Encyclopedia of Genes and Genomes (KEGG) Pathway Analysis generated by the Use case exemplified in the Examples, showing how succinate exposure differs from DCA exposure in increased cellobiose, fructoselysine (a fructooligosaccharide) and sorbitol phosphorylation. These differences can explain the mechanism of why succinate induces C. difficile to produce a significantly thicker biofilm. There are studies that have linked increase cellobiose, fructooligosaccharides and sorbitol metabolism and phosphorylation to succinate induced biofilm formation. The diverging regulation of mtIF, a gene involved in mannitol metabolism and phosphorylation, may be a testable difference between DCA and succinate induced biofilm formation. The downregulation of mtlF has been observed in succinate induced biofilms but the its levels have not been identified in DCA or bile acid induced biofilms.

### DETAILED DESCRIPTION OF THE INVENTION

As used herein, “subject” includes, but is not limited to, animals, plants, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In some forms, the subject can be any organism in which the disclosed method can be used to genetically modify the organism or cells of the organism.

The term “dashboard” as used herein, refers to a reporting mechanism that aggregates and displays metrics and key indicators so they can be easily accessed and viewed/examined by a user. In some forms, a dashboard is a graphical user interface, for example, as displayed on a video monitor connected with a computer, which provides at-a-glance views of key values/indicators relevant to a particular objective or process.

The term “inhibit” or other forms of the word such as “inhibiting” or “inhibition” means to decrease, hinder or restrain a particular characteristic such as an activity, response, condition, disease, or other biological parameter. It is understood that this is typically in relation to some standard or expected value, i.e., it is relative, but that it is not always necessary for the standard or relative value to be referred to. “Inhibits” can also mean to hinder or restrain the synthesis, expression or function of a protein relative to a standard or control. Inhibition can include, but is not limited to, the complete ablation of the activity, response, condition, or disease. “Inhibits” can also include, for example, a 10% reduction in the activity, response, condition, disease, or other biological parameter as compared to the native or control level. Thus, the reduction can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64,65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to native or control levels. For example, “inhibits expression” means hindering, interfering with or restraining the expression and/or activity of the gene/gene product pathway relative to a standard or a control.

“Treatment” or “treating” means to administer a composition to a subject or a system with an undesired condition (e.g., cancer). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount. “Prevention” or “preventing” means to administer a composition to a subject or a system at risk for an undesired condition (e.g., cancer). The condition can include one or more symptoms of a disease, pathological state, or disorder. The condition can also be a predisposition to the disease, pathological state, or disorder. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or reduction of the chances that a particular event or characteristic will occur.

As used herein, the terms “effective amount” or “therapeutically effective amount” means a quantity sufficient to alleviate or ameliorate one or more symptoms of a disorder, disease, or condition being treated, or to otherwise provide a desired pharmacologic and/or physiological effect. Such amelioration only requires a reduction or alteration, not necessarily elimination. The precise quantity will vary according to a variety of factors such as subject-dependent variables (e.g., age, immune system health, weight, etc.), the disease or disorder being treated, as well as the route of administration, and the pharmacokinetics and pharmacodynamics of the agent being administered.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

As used herein, the terms “variant” or “active variant” refers to a polypeptide or polynucleotide that differs from a reference polypeptide or polynucleotide, but retains essential properties (e.g., functional or biological activity). A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological or functional activity, certain amino acid sequence substitutions can be made in a polypeptide sequence and nevertheless obtain a polypeptide with like properties (e.g., functional or biological activity).

As used herein, “subject” includes, but is not limited to, animals, plants, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In some forms, the subject can be any organism in which the disclosed method can be used to genetically modify the organism or cells of the organism.

As used herein, the term “identity,” as known in the art, is a relationship between two or more nucleic acid or polypeptide sequences, as determined by comparing the sequences. In the art, “identity” also means the degree of sequence relatedness between nucleic acids or polypeptides as determined by the match between strings of such sequences. “Identity” can also mean the degree of sequence relatedness of a nucleic acid or polypeptide compared to the full-length of a reference nucleic acid or polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48:443-453, 1970) algorithm (e.g., NBLAST, and XBLAST). The default parameters are used to determine the identity for the nucleic acid or polypeptide sequence data of the present disclosure.

By way of example, a nucleic acid or polypeptide sequence may be identical to the reference sequence, that is be 100% identical, or it may include up to a certain integer number of nucleotide or amino acid alterations as compared to the reference sequence such that the % identity is less than 100%. Such alterations are selected from: at least one nucleotide or amino acid deletion, substitution, including conservative and non-conservative substitution, or insertion, and wherein said alterations may occur at the terminal positions of the reference nucleic acid or polypeptide sequence or anywhere between those terminal positions, interspersed either individually among the nucleotides or amino acids in the reference sequence or in one or more contiguous groups within the reference sequence. In some forms, the number of nucleotide acid or amino acid alterations for a given % identity is determined by multiplying the total number of amino acids in the reference nucleic acid or polypeptide by the numerical percent of nucleotides or amino acids or the respective percent identity (divided by 100) and then subtracting that product from said total number of nucleotides or amino acids in the reference nucleic acid or polypeptide.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

### I. Systems for Centralized Access to Gene Expression Datasets (CAT GxD)

A searchable bioinformatics analysis system has been developed for the integration of large datasets relating to host-pathogen interactions. An exemplary form of the system, known as Centralized Access to Gene Expression Datasets (“CAT-GxD”). has been developed using multi-omics data relating to the human pathogen Clostridioides difficile, however the system is amenable to the corresponding enhanced analysis and interpretation of datasets relating to other microorganisms, such as prokaryotes and viruses, for which a depot of data is available, or will become available, and also for higher organisms, such as multi-cellular animals.

In some forms, the system provides enhanced analysis and interpretation of gene expression data for cells, organs and/or tissues of animals, such as humans. In some forms, the system compares gene expression differences for any organism(s) for which suitable gene and/or protein expression data sets are available.

In exemplary forms, the CAT-GxD system provides analytical data for changes in gene expression in a host animal in response to a microbial infection. For example, in some forms, the CAT-GxD system provides analytical data for changes in gene expression in a host animal in response to a bacterial infection. In other forms, the CAT-GxD system provides analytical data for changes in gene expression in a host animal in response to a viral infection. Therefore, in some forms, the CAT-GxD system provides analytical data for changes in gene expression in a human in response to a microbial infection, such as a bacterial or viral infection. In an exemplary form, the CAT-GxD system provides analytical data for changes in gene expression in epithelial cells in response to a bacterial infection.

In some forms, the CAT-GxD system provides analytical data for changes in gene expression in diseased and/or damaged cells of an animal compared to healthy, control cells in the same or a different animal. For example, in some forms, the CAT-GxD system provides analytical data for changes in gene expression in cancer cells of an animal compared to normal, non-cancer cells in the same or a different animal. Therefore, in some forms, the CAT-GxD system provides analytical data for changes in gene expression in cancer cells in a human subject with cancer, as compared to normal, non-cancer cells in the same or a different human.

The CAT-GxD system is versatile and adaptable for different data input and output, according to the type of analyses requested. In some forms, the CAT-GxD system uses datasets including two or more types of information but lacking one or more other type of information. Therefore, in some forms, the CAT-GxD system provides output data for two or more types of information but lacking one or more other type of information. Typically, when the CAT-GxD system provides analytical data for changes in gene expression in eukaryotic cells and higher organisms, the system requires different. input data and may provide different or the same output data, as compared with when the system provides analytical data for changes in gene expression in eukaryotic cells or viruses. For example, in some forms, when the CAT-GxD system provides analytical data for changes in gene expression in eukaryotic cells based on datasets from eukaryotic cells, such as datasets for human cells including human expression data, the system will not include operon information in the Gene Info tab, consistent with the lack of operons in eukaryotic cells.

The described CAT-GxD system can be implemented on a computer, for example, including a user-interface for displaying and receiving inputs, such as a command or search term. In some forms, the computer-implemented methods include display of data in the format of a Dashboard, including an expandable format to include additional pathogens, and flexible capacity for integration of Principal Component analyses, experimental datasets and host-pathogen interaction prediction tools.

Methods of analyzing genetic sequences, for example comparative transcriptomics, proteomics, sequence homology, gene expression data, and/or pathogen host interactions using the CAT-GxD system are provided. In some computer implemented forms, the methods are carried out using a data processing algorithm such as a Principal Component Analysis (PCA). The methods provide a means of performing all-vs-all sequence analysis on large-scale data sets and use a combination of methodologies to interrogate sequence data, referred collectively as “data wrangling”, to consider genomic relatedness and differential gene expression data among multiple samples, and provide a structured database for visualization of data. The methods provide accurate, efficient, and scalable computation for comparative genomics that can be used to discern patterns in factors relating to a given pathogenic microorganism, such as epidemiology, virulence, host-pathogen relationships. and other factors. Typically, the input includes one or more pre-existing data sets, such as genomic, transcriptomic and/or proteomic data for two or more samples. Typically, the samples include two or more individuals in a population of microorganisms of the same species, optionally further including data from one or more hosts.

The resulting data can be utilized for methods of diagnosis, prognosis, and treatment of diseases and disorders associated with the pathogen.

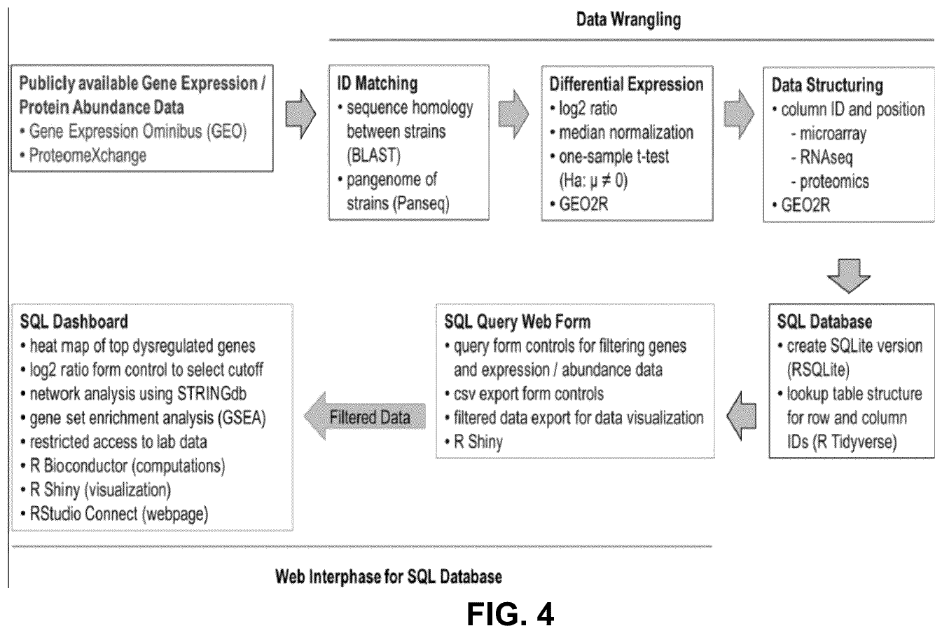

The methods can be expanded to the analysis of multiple databases, for example, to provide a quantitative comparison of two or more samples to rapidly compute functional differences in populations of microorganisms. For example, these analyses can facilitate understanding of how one or more factor(s) in a population of pathogens may change in response to one or more variable(s) such as geographic location, time, virulence, disease states of hosts, antimicrobial resistance, efficacy of vaccines and other interventions, etc. The methods can also provide predictions with respect to existing and future trends in microbial populations which can inform follow up analyses on the underlying biological mechanisms in pathogenic microorganisms that drive outbreaks of diseases, as well as epidemics and pandemics. A flow chart depicting an exemplary sequence of data wrangling and web interphase for the SQL database is set forth in FIG. 4.

In exemplary forms, the CAT-GxD system provides comparison of transcriptome and/or proteome alterations of one more organisms, such as microorganisms or host organisms, in response to one or more stimuli, or change in state, or genetic background. In some forms, the one or more stimuli include exposure to a specific compound, such as a drug or toxin. For example, in some forms, the CAT-GxD system provides a comparison of transcriptome and/or proteome alterations in response to exposure to an antibiotic. In other forms, the CAT-GxD system provides comparison of transcriptome and/or proteome alterations of one or more organisms in response to a genetic change, such as introduction of a specific mutation. In some forms, the output data include information about one or more cellular or gene regulatory elements, such as one or more regulatory checkpoints. Therefore, in some forms, the CAT-GxD system identifies and/or predicts the effect of post-transcriptional regulation of one or more genes and/or gene expression products. The methods integrate publicly available datasets of pathogen gene expression under different conditions. The methods can identify relevant gene expression data sets, conduct ID mapping between data sets, calculate expression and differential expression values and format these data such that they can be read by the R

Bioconductor package for subsequent visualization. An R Shiny app has also been developed. This can filter data based on gene descriptions and study type. Filtered data can be displayed as a heat map with gene and study ratio descriptions displayed by scrolling over a particular area of interest.

Typically the methods include:

i. Selecting an organism for analysis;

ii. providing input data in the form of two or more data sets containing input data relating to one or more of transcriptomic, genomic and/or proteomic data for a species or strain of organism;

iii. providing a relational database that combines data from the two or more datasets into a single, customized integrated and relational database using a suitable programming language, such as the structured query language (SQL);

iv. initiating an analysis of the integrated and relational database, for example using one or more input parameter including a keyword, gene identity, a gene-classification filter, an experiment-type filter and/or a fold-change cutoff parameter; and

v. providing an output of data from the analysis, for example, as a visual display, or in the form of an interactive dashboard showing indicated genes, and/or comparing expression levels, sequence homology, etc.

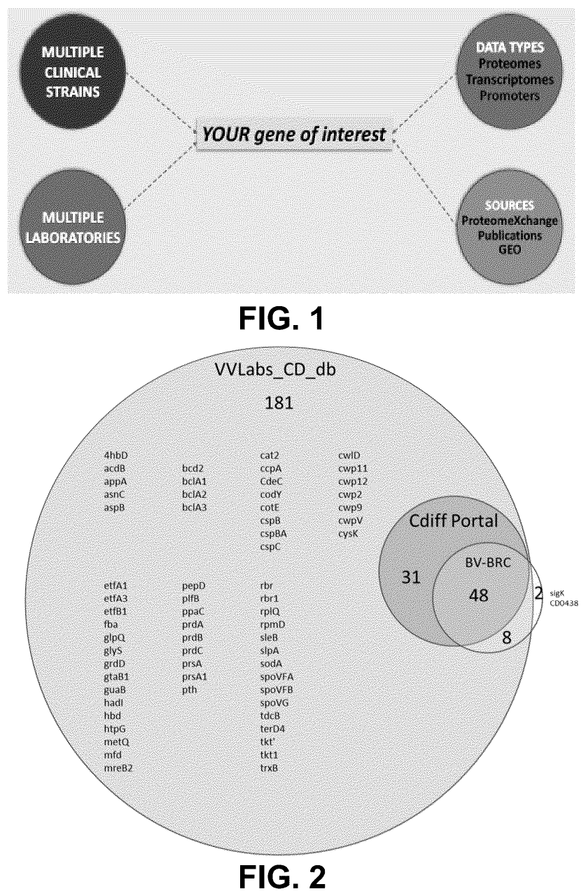

An exemplary workflow is illustrated in FIGS. 1-4, and each of the method steps is described in more detail, below.

### A. Selecting Source Organisms

The methods provide analyses for one or more selected organisms from which the input data are sourced (i.e., “selected source organism”). Therefore, any of the described methods can include one or more steps of selecting a source organism.

The methods can be configured to provide gene expression analyses for any organism or any subcomponent of an organism, such as a cell, tissue, organ, etc. for which suitable gene expression data are available.

In some forms, the source organism is a microorganism, such as a bacteria, fungi, virus or protozoan. In other forms, the source organism is an animal, such as a human. In some forms, the source animal is a host animal, such as an animal infected with one or more of a virus, bacteria, fungi, protozoan, a worm or other parasite. Therefore, in some forms the source organism is a human subject infected with one or more of a virus, bacteria, fungi, protozoan, a worm or other parasite. In other forms, the source organism is an animal suffering from a disease or disorder, such as a proliferative disease or disorder, such as a cancer. Therefore, in some forms, the source organism is a human subject suffering with a disease or disorder, such as a proliferative disease or disorder, such as a cancer. When the source organism is an animal having a disease or infection, the input data can include one or more of reference data sets obtained from the same or a different animal in the absence of the disease or infection. In some forms, when the source organism is an animal, the input data can be selectively derived from one or more type or group of cell, tissue or organ from the animal. Therefore, in some forms, the source is one or more specific cell, tissue or organs from an animal having a disease or disorder. When the source is one or more specific cell, tissue or organs from a an animal having a disease or infection, the input data can include one or more of reference data sets obtained from the same or a different animal in the absence of the disease or infection. For example, in some forms, the input data is derived from cancer cells of an human subject with cancer, and the input data optionally includes data derived from normal, non-cancer cells in the same or a different subject.

In other forms, the GxD system is adapted to a specific interaction between an organism, such as a microorganism and a host, for example, an interaction between a pathogenic bacterial species and a human host. For example, in some forms, a graphical interface includes a “Host-Pathogen GxD” tab. Therefore, in some forms, the system includes access to a data frame that includes specific genes in the host and/or pathogen that are known to be involved in the host-pathogen interaction.

### 1. Microorganisms

In some forms, the source organism is a microorganism, such as a single species, sub-species, clade or strain of bacteria, fungi, algae, archaea, parasite virus or protozoan. Typically, the source organism is an organism of significance to human health and/or industry, such as pathogenic microorganisms. In some forms, the microorganism is a fungi, such as a yeast or filamentous fungus. In other forms, the microorganism is an algae or a lichen. In some forms, CAT-GxD is adapted to a bacterium, such as a pathogenic bacterium.

Exemplary source bacteria include Staphylococcus aureus, E. coli, Lactobacillus Spp., Staphylococcus Spp., Streptococcus Spp., including Group A streptococcus, Bacillus Spp., Pseudomonas aeruginosa, Clostridium Spp., Mycobacterium Spp., Bacteroides Spp., Helicobacter pylori, Bifidobacterium Spp., Campylobacter Spp., Shigella Spp., Salmonella Spp., Neisseria Spp., Enterobacteriaceae, Haemophilus influenzae, and Oomycota Spp. In an exemplary form, CAT-GxD is used for analysis of the pathogenic bacterial species Clostridioides difficile or Neisseria gonorrhoeae.

Exemplary source fungi include Chytridiomycota, Zygomycota, Ascomycota, Basidiomycota, and Glomeromycota.

Exemplary source viruses include viruses that cause infection in animals, including Influenza, HIV, Human papillomavirus (HPV), Herpes, Rotavirus, Chicken pox, rhinovirus and coronaviruses, viruses that infect plants, such as Tobacco mosaic virus, Tomato spotted wilt virus, Tomato yellow leaf curl virus, Cucumber mosaic virus, Potato virus Y, Cauliflower mosaic virus, African cassava mosaic virus, Plum pox virus, Brome mosaic virus and Potato virus X, and viruses that infect bacteria, such as bacteriophage.

i. Pathogenic Microorganisms

In some forms, the microorganism is a pathogenic microorganism. Exemplary microorganisms include bacteria, viruses, fungi and protozoa.

In some forms, the microorganism is a pathogenic microorganism that causes diseases and disorders in animals, such as humans. Therefore, in some forms, the pathogenic microorganism causes disease and/or disorders in animals such as humans. Pathogenic microorganisms that cause disease and/or disorders in animals such as humans, and which are resistant to standard-of care practices for treatment are recognized as considered an Urgent Threat to US healthcare by the CDC. Exemplary pathogenic microorganisms include antibiotic-resistant bacteria, such as drug-resistant Campylobacter, drug-resistant Candida Species, ESBL-producing Enterobacterales Vancomycin-resistant Enterococcus (VRE), Multidrug-resistant Pseudomonas aeruginosa, Drug-resistant nontyphoidal Salmonella, Drug-resistant Salmonella serotype Typhi, Drug-resistant Shigella, Methicillin-resistant Staphylococcus aureus (MRSA), Drug-resistant Streptococcus pneumoniae and Drug-resistant Tuberculosis. In some forms, the pathogenic microorganism is a pathogenic bacteria, such as Clostridioides difficile, Erythromycin-resistant Group A Streptococcus (GAS), Clindamycin-resistant Group B Streptococcus (GBS), Drug-resistant Mycoplasma genitalium (M. genitalium), Drug-resistant Bordetella pertussis (B. pertussis), or a pathogenic fungi, such as Azole-resistant Aspergillus fumigatus.

In some forms, the pathogenic microorganism is selected from Clostridioides spp., Neisseria spp., Candida spp., Enterobacteriaceae spp., Acinetobacter spp., Campylobacter spp., and Escherichia spp. For example, in some forms, the pathogenic microorganism is selected from Clostridioides difficile, Neisseria gonorrhoeae, Candida auris, and Escherichia coli. 9l. In some forms, the pathogenic microorganism is an antibiotic-resistant bacterium such as a carbapenem-resistant Enterobacteriales sp. or a carbapenem-resistant Acinetobacter sp.

a. Clostridioides difficile

In some forms, the pathogenic microorganism is the pathogenic bacteria Clostridioides difficile.

C. difficile is a Gram-positive, anaerobic, spore-forming bacillus that causes (sometimes fatal) diarrhea via the production of 1-3 toxins. Non-toxin factors are also critical for disease establishment and persistence. C. difficile is an important pathogen of both human and veterinary populations, with non-human neonates (piglets, calves and foals) being particularly affected, and susceptible to C. difficile infection (CDI) within 1-14 days of birth. In swine operations, the greatest numbers of C. difficile isolates are recovered from suckling piglets inside the farrowing barn, and on some farms, >70% of all piglets carry C. difficile, and a subset will succumb to the disease. Since the immune system is under-developed in neonates, classic approaches such as vaccination are not used for CDI prevention.

C. difficile veterinary strains have also been recovered from human patients, leading to the contention that farms/food may be reservoirs of the pathogen. Surveillance and monitoring research reveals that identical C. difficile molecular types are prevalent in both agricultural, and human healthcare, settings.

Antibiotics remain the mainstay of CDI treatment (ceftiofur, enrofloxacin, apramycin), but due to their attendant dysbiosis, further delay the development of a healthy microbiota. Patients/animals that survive infection can become asymptomatically colonized, and shed C. difficile to further contaminate the environment. There have been multiple high-profile failures in clinical trials focused on vaccines and anti-infectives.

C. difficile is considered an Urgent Threat to US healthcare by the CDC (Finn, et al., 2021). Annual cases range from 500,000-1,000,000 resulting in ˜29,000 deaths and >$6.3 billion cost overall to healthcare systems. There are currently no vaccines against C. difficile infections, and there are several limitations to antibiotic therapy, including the emergence of resistant strains.

The mechanisms by which C. difficile causes disease are poorly understood and are an active area of investigation. The ˜4,000 genes of C. difficile are controlled by complex regulatory networks that are responsive to metabolic and environmental cues; the functions of most of these genes in bacterial physiology and virulence remain undefined.

b. Neisseria gonorrhoeae

In some forms, the pathogenic microorganism is the pathogenic bacteria Neisseria gonorrhoeae. N. gonorrhoeae, also known as gonococcus (singular) or gonococci (plural), is a species of Gram-negative diplococci that causes the sexually transmitted genitourinary infection gonorrhea in humans, as well as other forms of gonococcal disease including disseminated gonococcemia, septic arthritis, and gonococcal ophthalmia neonatorum. N. gonorrhoeae infects the mucous membranes of the reproductive tract, including the cervix, uterus, and fallopian tubes in women, and the urethra in women and men, and can also infect the mucous membranes of the mouth, throat, eyes, and rectum, and can be spread perinatally from mother to baby during childbirth. CDC estimates that approximately 1.6 million new gonococcal infections occurred in the United States in 2018, and more than half occur among young people aged 15-24.

N. gonorrhoeae, is oxidase positive and aerobic, and survives phagocytosis to grow inside neutrophils. It exhibits antigenic variation through genetic recombination of its pili and surface proteins that interact with the immune system. Sexual transmission is through vaginal, anal, or oral sex. Sexual transmission may be prevented through the use of barrier protection.

N. gonorrhoeae can cause infection of the genitals, throat, and eyes, though, asymptomatic infection is common. Untreated infection may spread to the rest of the body (disseminated gonorrhea infection), especially the joints (septic arthritis). Untreated infection in women may cause pelvic inflammatory disease and possible infertility due to the resulting scarring.

### 2. Host-Pathogenic Interactions

In some forms, the GxD system is adapted to a specific interaction between an organism, such as a microorganism and a host, for example, an interaction between a pathogenic bacterial species and a human host. For example, in some forms, a graphical interface includes a “Host-Pathogen GxD” tab. Therefore, in some forms, the system includes access to a data frame that includes specific genes in the host and/or pathogen that are known to be involved in the host-pathogen interaction.

A pathogen interacts with the host and causes infection, leading to the development of disease in the host. A pathogen may be any harmful microbial agent such as a bacterium, virus, protozoa, fungus or helminth etc. When the pathogen enter the host cell, it has to face strong panoply of immune defense system inside, that further confine and eliminate the pathogen. At the interface of host-pathogen interaction, when a pathogen-ligand interacts with its specific host cell receptor, it results in its activation and ultimately leads to the recruitment of signaling molecules via signaling cascades. In immune cells, signal transduction cassettes consist of specific surface bound membrane receptors like B-cell receptors, T-cell receptors, co-stimulatory receptors and cytokine receptors, that on activation lead to recruitment of various regulatory proteins and effector signaling elements. These cassettes detect, amplify and integrate the external signals generated from ligand-receptor binding to trigger the appropriate and adequate effector responses of the immune system in order to achieve complete removal of pathogens and limit the host damage to the minimum. However, some of the pathogens utilize these communication pathways as key targets to modulate the host immune response and promote their survival and multiplication. In some forms, the methods identify and/or connect one or more pathogen factors, such as gene expression, proteome, phenotypic factors, etc., with one or more host factors, such as genomic, proteomic or phenotypic factors. In some forms, the methods analyze host-pathogen interactions to provide a better understanding of the infectious diseases and various mechanisms being adopted by the pathogens to cause infection. In some forms, the methods provide new discernment about the elemental aspects of microbial pathogenicity and assist development of better treatment and prevention of infectious diseases.

Exemplary pathogens include pathogenic microorganisms, such as bacteria, viruses, fungi, and protozoa. Exemplary hosts include animals, such as farm animals, birds, fish and primates. Exemplary hosts include humans.

### B. Providing Input Data

The methods include one or more steps of providing input data relating to the source organism(s) for analyses.

The described multi-omics CAT-GxD systems typically include comparing DNA, RNA and/or protein samples collected from a multiplicity of pathogenic microorganisms of the same species and/or subtype and/or clade. Typically, the methods are implemented on a computer, using a computer program. In some forms, multi-omics data for a multiplicity of microorganisms of the same species is provided in the form of a database. In some forms, databased information from one or more host organisms is also provided. Exemplary forms of data include sequence data, such as the sequence of nucleotides within nucleic acids such as DNA and RNA, or the sequence of amino acids of or within polypeptides. In some forms, the input data are non-sequence-based data, such as structural data or other data relating to a sample. In some forms, the data are provided in a computer-readable format. Therefore, in some forms, the data are provided in the form of a computer-readable database. For example, the input data may be converted from a sequence format into a binary or other computer-readable format. An exemplary computer readable format is STRING text used as an input for the computer program language PYTHON.

In molecular biology, STRING (Search Tool for the Retrieval of Interacting Genes/Proteins) is a biological database and web resource of known and predicted protein-protein interactions. The STRING database contains information from numerous sources, including experimental data, computational prediction methods and public text collections. It is freely accessible and it is regularly updated. The resource also serves to highlight functional enrichments in user-provided lists of proteins, using a number of functional classification systems such as GO, Pfam and KEGG. The latest version 11b contains information on about 24.5 million proteins from more than 5000 organisms. STRING has been developed by a consortium of academic institutions including CPR, EMBL, KU, SIB, TUD and UZH.

Therefore, in some forms, the data is provided in the form of a publicly available database. Multiple extensive multi-omic bacterial gene expression datasets are publicly available. Publicly available bacterial pathogen gene expression datasets include transcriptomic (RNAseq and Microarray) and proteomics data, as well as regulatory and structural information. Exemplary samples include diverse clinical isolates, specific mutant derivatives, and bacteria grown under various different conditions.

In some forms, the input data includes data from one or more than one source, such as from 2 or more, 3, 4, 5, 6, 7, 8, 9, 10, 20, 30, 40, 50, 60, 50, 80, 90, 100, or more than 100, such as 200, 500 or 1,000 different sources or databases. Exemplary data sources and inputs include the Rapid Annotations using Subsystems Technology (RAST) subsequence database (Aziz et al., BMC Genomics. 2008; 9:75); the KEGG Database (Kanehisa, et al., Protein Sci. 31, 47-53 (2022); a Clusters of Orthologous Groups (COG) database (Tatusov, et al., Nucleic Acids Res. 2000 Jan. 1; 28 (1): 33-36); a gene ontology (GO) database (Young, et al., Genome Biology volume 11, Article number: R14 (2010));

and different enzyme classes, as well as experimental filters (i.e., omics-type of data, as well as stress and knockout profiling data), BioCyc, The Database for Annotation, Visualization and Integrated Discovery (DAVID) DAVID bioinformatics database, UniProt, and Gene Expression omnibus (GEO) databases.

In some forms, the input data includes data from the BioCyc Genomic database collection. BioCyc is a collection of 20,050 Pathway/Genome Databases (PGDBs) for model eukaryotes and for thousands of microbes, plus software tools for exploring them. BioCyc is an encyclopedic reference that contains curated data from 146,000 publications. BioCyc includes data on phenotypic properties of organisms, including human-microbe body sites, aerobicity and optimal temperature ranges for microbial physiological functions.

In some forms, the input data includes data from the Database for Annotation, Visualization and Integrated Discovery (DAVID) bioinformatics database. DAVID provides a comprehensive set of functional annotation tools for investigators to understand the biological meaning behind large lists of genes. These tools are powered by the comprehensive DAVID Knowledgebase built upon the DAVID Gene concept which pulls together multiple sources of functional annotations.

In some forms, nucleic acid (e.g., DNA) data includes classifications, such as gene classifications. Typically, gene classifications are allocated and defined by one or more publicly available data acquisition and archiving initiatives, including bioinformatics organizations such as the National Center for Bioinformatics (NCBI), European Bioinformatics Institute (EBI), Kyoto Encyclopedia of Genes and Genomes (KEGG), and Gene Ontology. The systems incorporate such classifications as tags for DNA data in the input data. Typically, gene classification data is used as a filter to simplify, order, and/or focus the input data into an output dataset, for example, data that can be visualized as one or more of a heatmap, PCA and network. In some forms, gene input data that is used for data filtering includes results of computation programs. For example, in some forms, the system filters gene input data using the program PSORTb to determine protein localization and/or genetic assay results, where available. For example, in an exemplary form, gene input data includes gene data for the microorganism C. difficile, filtered using the program PSORTb to identify protein localization and genetic assay data from the study of Milton et. al., to determine gene essentiality.

In some forms, the input data that includes RNA expression data includes a first data set including gene annotations as reference IDs, and a second or further data sets that each include one or more gene IDs of other organisms strains that are mapped to the reference IDs based on gene (e.g., nucleic acid and/or amino acid sequence) homology between the first and second or further data sets. In some forms, the methods include one or more steps to calculate sequence homology according to an algorithm for comparing primary biological sequence information. An exemplary algorithm is implemented within the publicly available computer software “Basic Local Alignment Search Tool” (BLAST). In an exemplary form, sequence homology data are calculated and incorporated into a database that is directly imported into the system. In some forms, the methods apply one or more additional modifications to sequence homology datasets, for example, to remove replicate IDs and/or duplicate experiments.

Typically, the input data is or includes values for one or more pieces of data such as sequence data for DNA (e.g., genomic data), RNA (e.g., transcriptomic data) or protein (e.g., proteomic data) for one or more select source organism(s). Input data can be in the form of raw (i.e., unprocessed) or computed (i.e., processed) data. When one or more input datum/data are provided in completely unprocessed or partially unprocessed form, the methods optionally include one or more modifications of the one or more input datum/data, to provide completely processed input data. For example, in some forms, the methods include one or more steps of processing data to comply with a format that can be interpreted by the CAT-GxD system. In some forms, one or more modifications includes editing of the data using any suitable means known in the art for manipulating data, such as using the Excel and GEO2R programs.

In some forms, the input data is controlled, selected or otherwise determined by the use of one or more organism-specific gene filters. In some forms, the available filters (KEGG, COG, etc) were designed for non-microbial organisms, including human genomes. Therefore, in some forms, these databases have limited utility for a pathogenic microorganism, such as C. difficile, or N. gonorrhoeae. Therefore, in some forms, the step of providing input data includes selecting or applying one or more organism-specific filters that are tailored to the target organism. In an exemplary form, providing input data for the target organism C. difficile includes the use of one or more C. difficile-specific gene filters. In another exemplary form, providing input data for the target organism N. gonorrhoeae includes the use of one or more N. gonorrhoeae-specific gene filters.

### 1. DNA/genomic Data

In some forms, the input data includes nucleic acid sequence data corresponding to DNA from a sample. Typically, the DNA is genomic DNA. In some forms, when the DNA is genomic DNA, the data includes DNA corresponding to all of the genome of the microorganism. In other forms, a sample includes less than the entire genome, such as one or more fragments of a genome.

DNA is a double helix structure composed of two strands that are complements of each other. A strand is a sequence that is made up of one or more of four different characters, ‘A’,'T′,'G′ and ‘C’, representing the four nucleobases adenine, thymine, guanine and cytosine, respectively. ‘A’-'T′ and ‘G’-'C′ are paired in the complementary strands. Each strand has an orientation, i.e. ‘5’-end to 3′-end, and complementary strands have opposite orientations. The sequence of A, T, G, and C nucleobases in a strand of DNA is read by sequencing machines, and is called a read. There is a limit, however, to the number of sequential nucleobases that current sequencing technology can produce, which given some current technologies can be a length of a few hundred nucleobases. When computing the entire DNA sequence for a pathogenic organism these limitations are overcome by producing many overlapping reads of the DNA, then stitching them together to produce the entire DNA sequence. The sequence can be read in two ways, forward and reverse-complement, each representing a particular strand of DNA.

### 2. RNA/transcriptomic Data

In some forms, the input data includes nucleic acid sequence data corresponding to RNA from a sample. The analysis of RNA/transcriptomic data is indicative of the expression of specific genes or gene subsets within the sample. Exemplary forms of transcriptomic data include RNAseq and Microarray data.

RNA is a double helix structure composed of two strands that are complements of each other or, alternatively, exists as a single strand. A strand is a sequence that is made up of one or more of four different characters, ‘A’,'U′,'G′ and ‘C’, representing the four nucleobases adenine, uracil, guanine and cytosine, respectively. ‘A’-'U′ and ‘G’-'C′ are paired in the complementary strands. Uracil replaces Thymine in RNA. Each strand has an orientation, i.e., ‘5’-end to 3′-end, and complementary strands have opposite orientations. The sequence of A, U, G, and C nucleobases in a strand of RNA is read by sequencing machines, and is called a read.

In some forms, RNA is converted to complementary DNA (“cDNA”) prior to sequencing and incorporation into a database. cDNA is typically produced by the activity of reverse transcriptase enzyme. An exemplary cDNA is synthetic DNA that has been transcribed from a specific mRNA through a reverse transcriptase reaction. Therefore, in some forms, the input data includes nucleic acid sequence data corresponding to cDNA, corresponding to RNA from a sample.

In some forms, RNA expression input data is from NCBI's Gene Expression Omnibus (GEO) database. When RNA expression input data is from the GEO database, the data typically include microarray and RNA sequences for an organism or cell or tissue sample.

In an exemplary form, the RNA expression input data includes C. difficile strain 630 gene annotations as reference IDs, whereby gene IDs of other C. difficile strains are mapped to strain C. difficile 630 based on sequence homology data calculated by an algorithm for comparing primary biological sequence information. An exemplary algorithm is implemented within the publicly available computer software “Basic Local Alignment Search Tool” (BLAST). In an exemplary form, RNA homology data are calculated and incorporated into a database that is directly imported into the system. In some forms, the methods apply one or more additional modifications to one or more RNA homology dataset(s), for example, to remove replicate IDs and/or duplicate experiments. In some forms, the input data includes or is raw values for RNA or amino acids sequence. Therefore, in some forms the methods include one or more modifications of the input RNA data to comply with a format that can be interpreted by the program. Exemplary modifications include editing of the data using the Excel and/or GEO2R programs.

### 3. Protein/Proteomic Data

In some forms, the input data includes amino acid sequence data corresponding to proteins/proteomics analyses from one or more samples.

As used herein, the term “polypeptides” includes proteins and functional fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). Certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties. The hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within +2 is preferred, those within +1 are particularly preferred, and those within +0.5 are even more particularly preferred.

Substitution of like amino acids can also be made on the basis of hydrophilicity, particularly, where the biological functional equivalent polypeptide or peptide thereby created is intended for use in immunological forms. The following hydrophilicity values have been assigned to amino acid residues: arginine (+3.0); lysine (+3.0); aspartate (+3.0 +1); glutamate (+3.0 +1); serine (+0.3); asparagine (+0.2); glutamine (+0.2); glycine (0); proline (−0.5 +1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within +2 is preferred, those within +1 are particularly preferred, and those within +0.5 are even more particularly preferred. As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly:

Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Forms of this disclosure thus contemplate functional or biological equivalents of a polypeptide as set forth above.

In particular, forms of the polypeptides can include variants having about 50%, 60%, 70%, 80%, 90%, and 95% sequence identity to the polypeptide of interest.

The analysis of protein abundance is indicative of the expression and translation of specific genes or gene subsets within the sample. In some forms, the protein/proteomics data include data relating to post-translational modifications of one or more proteins within the dataset. In other forms, the protein/proteomics data do not include data relating to post-translational modifications of one or more proteins within the dataset.

In an exemplary form, the methods provide quantitative proteomic input data obtained from the European Bioinformatics Institute (EBI) Proteome Xchange database. In a particular form, the methods provide quantitative proteomic input data for C. difficile strain 630 gene annotations for use as reference IDs, and gene IDs of other C. difficile strains are mapped to strain 630 based on sequence homology, for example, as determined by BLAST. In some forms, the methods include one or more steps including providing computed quantitative proteomic input datasets and inputting these datasets directly into the system. Datasets that need additional computation due to replicate IDs and/or experiments, and if only raw values were submitted, are finalized using Excel.

4. Exemplary C. difficile Data

In exemplary forms, the selected source organism is C. difficile, and the input data include C. difficile-specific datasets, including data for C. difficile gene expression and/or C. difficile protein quantitation.

In some forms, C. difficile-specific gene expression is obtained from a publicly-available database that presents C. difficile expression data.

As of December 2023, publicly available datasets of C. difficile gene expression under different conditions include 86 transcriptomics series datasets from the Gene Expression Omnibus (GEO) database and 27 proteomics data series from ProteomeXchange Consortium. No other C. difficile proteomics analysis resource is currently available. However, there are two existing bioinformatics resources, the Bacterial and Viral Bioinformatics Research Center (BV-BRC) and C. difficile Portal, that aid in mining C. difficile transcriptomics datasets.

BV-BRC, formed in 2019 under the National Institute of Allergy and Infectious Diseases (NIAID) Bioinformatics Resource Center (BRC) program, provides access to multiple bioinformatic analysis tools to mine genomic and transcriptomics data of viral and bacterial pathogens including C. difficile. BV-BRC (version 3.30.19a) includes 4 transcriptomics datasets, submitted to GEO database between 2010-2011.

Leveraging on 11 transcriptomics datasets submitted to GEO database from 2010-2018, the C. difficile Portal was launched in November 2021 to aid in predicting regulatory and metabolic network models for C. difficile and assigning functions to uncharacterized genes using two computational modeling algorithms, the environment and gene network (EGRIN) and the phenotype of regulatory influences integrated with metabolism and environment (PRIME). Both BV-BRC and C. difficile Portal focused on the analyzing transcriptomics datasets derived from C. difficile strain 630.

While C. difficile 630 has been widely used for various mechanistic studies, the genetic and phenotypic diversity, particularly of current clinical strains, is well recognized and publicly available transcriptomics and proteomics datasets were generated with at least 5 different C. difficile strains, and cognate mutant derivatives.

The methods can employ, e.g., the C. difficile strain 630 as a reference strain. Therefore, in some forms, protein identity and gene position comparisons are conducted based on the reference strain C. difficile strain 630. Gene classifications were compiled to provide a comprehensive and robust search.

### C. Providing an Integrated Relational Database

In some forms, the methods include one or more steps of conforming multiple datasets into an integrated relational database amenable to subsequent analysis.

Typically, the input data includes two or more datasets. For example, in some forms, the input data includes two or more databases. When multiple databases are provided, the databases can be in the same or different format. Therefore, in some forms, the methods include one or more steps of combining and conforming data from two or more datasets or data bases with the same or different data formats, into a single format that enables direct comparisons between data in the two or more datasets. In some forms, the methods combine data from two or more than two different sources or databases, such as 3 or more different sources or databases, 4, 5, 6, 7, 8, 9, 10, 20, 30, 40, 50, 60, 50, 80, 90, 100, or more than 100 sources, such as 200, 500 or 1,000 different sources or databases.

Typically, the step of conforming data from distinct sources and/or formats does not remove or change the values of the relevant data in the two or more datasets. For example, in preferred forms, conforming the data maintains the values for factors such as gene expression, abundance and sequences as constant before and after the formatting. Typically, the methods create a relational database as a collection of highly structured tables, e.g., wherein each row reflects a data entity, and every column defines a specific information field. Relational databases are built using the structured query language (SQL) to create, store, update, and retrieve data. Integrated relational databases including a collection of highly structured tables, wherein each row reflects a data entity, and every column defines a specific information field are described.

In some forms, the methods of conforming datasets include one or more of the steps of sequence identity matching, determining differential gene expression and data structuring.

### 1. Identity Matching

The methods typically include the step of sequence identity matching. In some forms, the step of sequence identity matching includes determining sequence homology and/or identity between two or more samples, for example, using the alignment program BLAST. In some forms, the step of sequence identity matching includes determining pangenome between two or more samples, for example, using the alignment program Panseq. The pan-genome of a bacterial species includes a core and an accessory gene pool. The accessory genome is thought to be an important source of genetic variability in bacterial populations and is gained through lateral gene transfer, allowing subpopulations of bacteria to better adapt to specific niches. Low-cost and high-throughput sequencing platforms have created an exponential increase in genome sequence data and an opportunity to study the pan-genomes of many bacterial species. Panseq determines the core and accessory regions among a collection of genomic sequences based on user-defined parameters. It readily extracts regions unique to a genome or group of genomes, identifies SNPs within shared core genomic regions, constructs files for use in phylogeny programs based on both the presence/absence of accessory regions and SNPs within core regions and produces a graphical overview of the output. Panseq also includes a loci selector that calculates the most variable and discriminatory loci among sets of accessory loci or core gene SNPs.

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48:443-453, 1970) algorithm (e.g., NBLAST, and XBLAST). The default parameters are used to determine the identity for the polypeptides of the present disclosure.

By way of example, a nucleic acid or polypeptide sequence may be identical to the reference sequence, that is be 100% identical, or it may include up to a certain integer number of nucleotides or amino acid alterations as compared to the reference sequence such that the % identity is less than 100%. Such alterations are selected from: at least one nucleotide or amino acid deletion, substitution, including conservative and non-conservative substitution, or insertion, and wherein said alterations may occur at the 5′ or 3′ or amino-or carboxy-terminal positions of the reference nucleic acid or polypeptide sequence or anywhere between those terminal positions, interspersed either individually among the nucleotides or amino acids in the reference sequence or in one or more contiguous groups within the reference sequence. In some forms, the number of position alterations for a given % identity is determined by multiplying the total number of positions in the reference sequence by the numerical percent of the respective percent identity (divided by 100) and then subtracting that product from said total number of positions in the reference sequence.

In some forms, default parameters can be used to determine the identity for the sequences of the present disclosure. In some forms, the % sequence identity of a given nucleic acid sequence or amino acid sequence “C” to, with, or against a given nucleic acid or amino acid sequence “D” (which can alternatively be phrased as a given sequence C that has or includes a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides in D. It will be appreciated that where the length of sequence C is not equal to the length of sequence D, the % sequence identity of C to D will not equal the % sequence identity of D to C.

### 2. Determining Differential Gene Expression

The methods typically include the step of determining differential expression. In some forms, the step of sequence determining differential expression includes one or more of determining of the log2 ratio between samples, median normalization, one-sample t-test and expression analysis using the R Bioconductor program suite.

In some forms, the analysis includes comparison of the expression of the same gene or group of genes across multiple samples. Therefore, in some forms, the methods include one or more steps for quantifying and comparing gene expression. In some forms, gene expression is depicted in the format of a gene expression “value” or “score” that is relative to the expression of the same gene or group of genes from one or more other samples. In some forms, the expression score is based on a control value, for example, the expression of the same gene in the same by a reference microorganism under the same conditions. There are multiple means known in the art for calculating and comparing gene expression in one or more samples. In some forms, expression is calculated using a principle component analysis (PCA)-based algorithm.

i. Log2 Transformation of Gene expression Data

In some forms, the methods transform gene expression data using one or more Log2 transformations.

Log2 aids in calculating fold change, and up-regulated vs down-regulated genes between replicates/samples, using base-2 ratios computed as the log 2 transform of the ratio between the test (numerator) and the control (denominator). Typically, Log2 transformations of input data include a log2 ratio computation, then perform a normalization calculation and, compile the data. Therefore, in some forms, for each two or more input datasets, the methods include one or more steps of log2 ratio computation, normalization and, compilation. Median normalization is typically performed on all gene log2 ratios resulting in a dataset median of 0.

In some forms, the methods compute gene to gene log2 ratios for the same or different gene or groups of related genes for a given source organism in two or more different conditions (e.g., experiments, environments, genetic backgrounds, disease states, etc.) as the primary comparative analysis.

Therefore, in some forms, the methods calculate log2 ratios that represent the expression of one or more genes under a specific, known set of differing conditions. Therefore, in some forms, the methods provide a log2 ratio for gene expression for a given pathway or cellular process under a specific, known set of differing conditions. In an exemplary form, the methods provide a log2 ratio for one or more expressed genes correlated with the presence or absence of an environmental or cellular stimulus. An exemplary stimulus is the presence of an antimicrobial agent.

ii. Computation of Gene expression Data: R Bioconductor

In some forms, the input data is configured for computation using the R Bioconductor computer software. R Bioconductor is an open source and open development software project for the analysis and comprehension of genomic data generated by wet lab experiments in molecular biology. R Bioconductor is based primarily on the statistical R programming language, but does contain contributions in other programming languages. See, Bioconductor website, e.g., Bioconductor.org.

iii. Computation of Gene Expression Data: GEO2R

In some forms, the input data is structured by the GEO2R computer software. GEO2R is an interactive web tool that allows users to compare two or more groups of Samples in a GEO Series in order to identify genes that are differentially expressed across experimental conditions. Results are presented as a table of genes ordered by significance, and as a collection of graphic plots to help visualize differentially expressed genes and assess data set quality. See, e.g., Gene Expression Omnibus page at the NCBI website, e.g., ncbi.nlm.nih.gov.

### 4. Data Structuring

The methods typically include one or more steps of structuring the identity-matched data within a relational database.

Many publicly available bioinformatics repositories, such as NCBI and EBI, include data having different formats for reporting fold-change and/or raw expression values in GEO and ProteomeXchange, respectively. Therefore, in some forms, input data have to be manually formatted in order for log2 ratio computation, normalization, and compilation.

In an exemplary form, as depicted in the Examples, below, a C. difficle strain 630 gene annotations were used as reference IDs to which other C. difficile strains' gene IDs were mapped based on BLAST homology results. Datasets already computed were directly imported into the system. Datasets that needed additional computation due to replicate IDs and/or experiments, and if only raw values were submitted, were finished using Excel and GEO2R programs.

Typically, the methods combine and/or format the input data into a relational data base that is constructed using a suitable computer programming language. In some forms, the relational database is built using the structured query language (SQL) to create, store, update, and retrieve data. In other forms, the system does not include SQL, but implements an alternative programming language having the same or similar functionality as SQL.

i. Structured Query Language (SQL) Data

SQL is a programming language synonymous with relational databases. The SQL language implements a stable and effective means to manage, manipulate and view data.

A SQL database or relational database is a collection of highly structured tables, wherein each row reflects a data entity, and every column defines a specific information field. Therefore, integrated relational databases including a collection of highly structured tables, wherein each row reflects a data entity, and every column defines a specific information field built using the structured query language (SQL) are described.

Typically, data structuring includes creating a column ID and position for data including e.g., microarray data, RNAseq data, and proteomics data.

In an exemplary form, as demonstrated in the examples, the methods conform data from 41 separate nonredundant GEO transcriptomics datasets and 11 separate proteomic datasets into a single SQL database. In some forms, the step of preparing a relational database includes one or more steps to identify and remove duplicate data within a multiplicity of datasets. In some forms, the methods filter and remove one or more data points according to the requirements of the analysis. For example in some forms, proteomics datasets are filtered to include total proteomic analyses and to remove post-translational modification data. Typically, the identity of genes is maintained according to, or otherwise matched to, the nomenclature of a widely accepted reference genome.

### D. Examining Integrated Relational Data

The methods include one or more steps of analyzing the integrated relational database. Typically, the analyzing includes one or more steps of screening, comparing, searching or otherwise filtering the data within the integrated relational database. The methods initiate the analysis based on one or more user-defined input or search parameters that form the basis of the alignments and/or analyses that are to be reported. Therefore, the methods include analyzing the data based on a received input from the user.

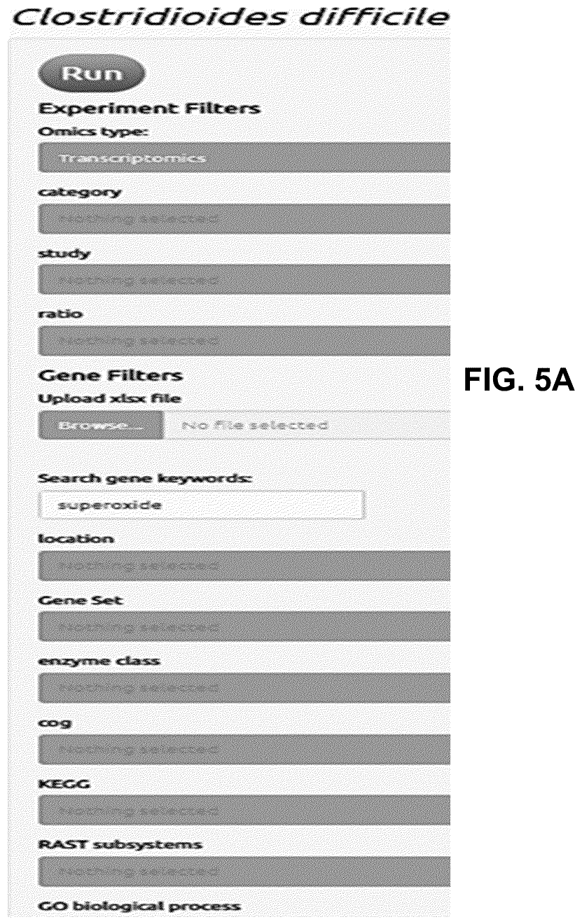

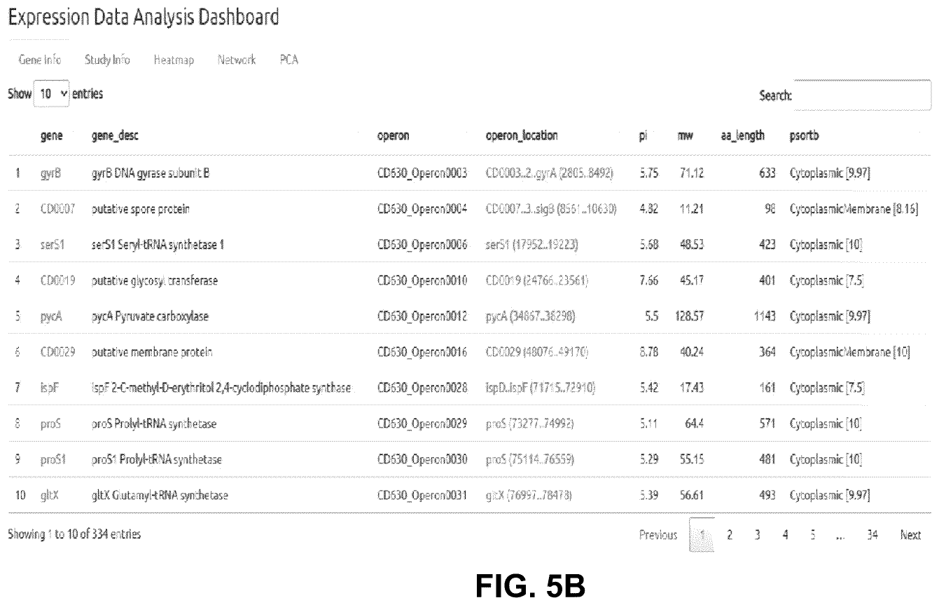

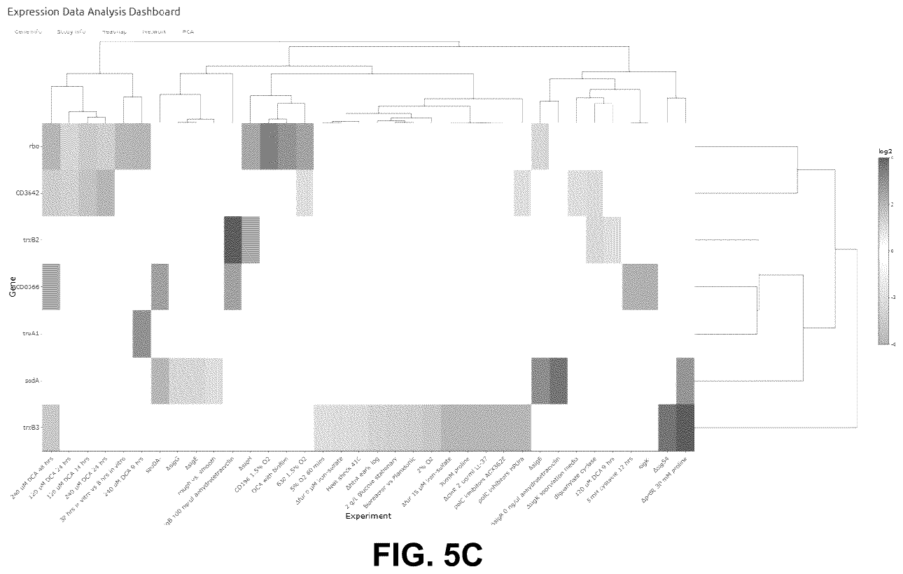

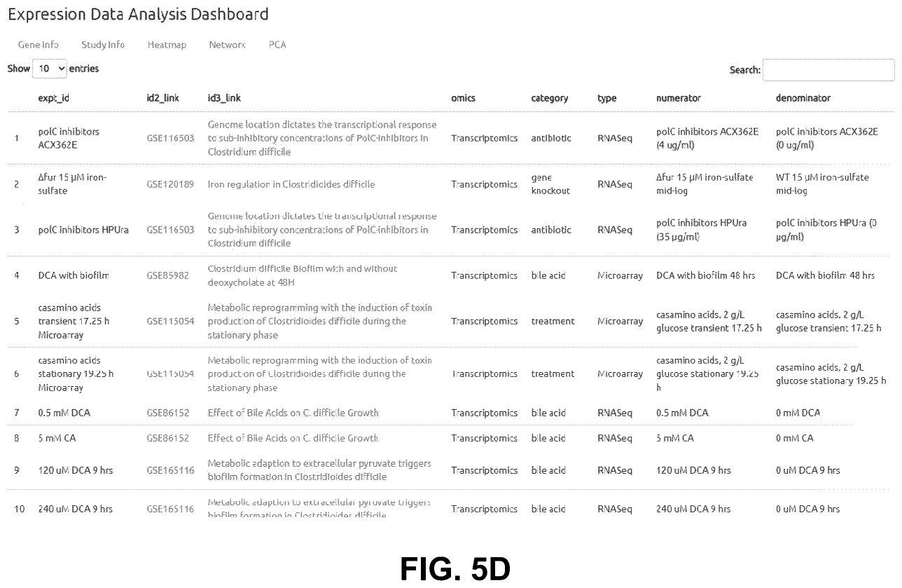

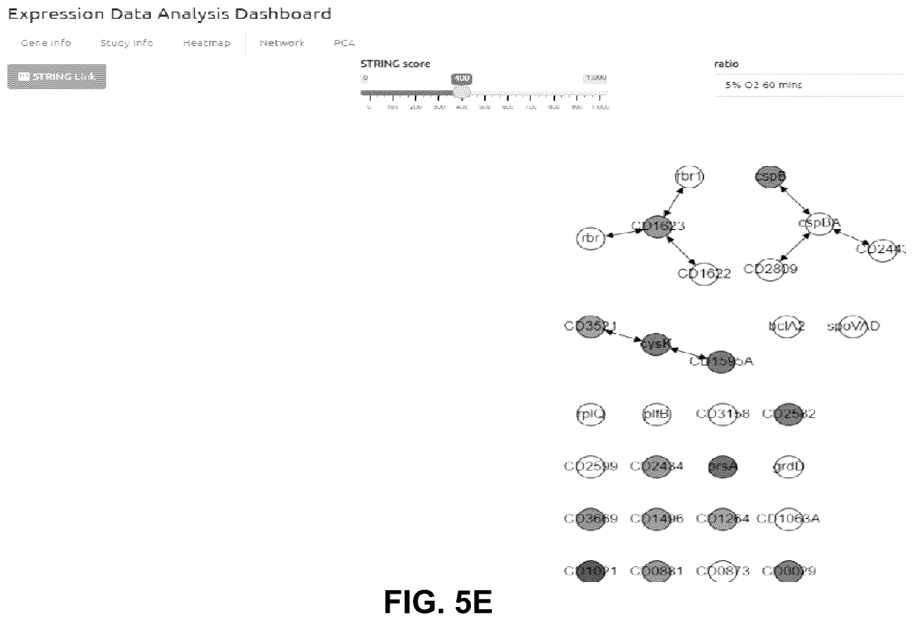

Typically, when the methods are implemented on a computer using a computer program, the computer program is controlled by a user entering one or more inputs through a general-user interface (GUI), such as a “dashboard” display. In some forms, the computer program is implemented through an internet-accessible platform. In some forms, the analyses includes determining and comparing the expression of the one or more genes, for example, by accessing data for quantitative proteomic analysis and/or transcriptomic analysis. Therefore, in some forms, input parameters include identifying or selecting one or more genes based on a classification, for example, as part of the pangenome, core genome, metabolic core genome, and/or essential genes. In some forms, input parameters include identifying or selecting one or more genes based on its cellular localization, such as the cytoplasm or within cytoplasmic membrane. In some forms, input parameters include identifying or selecting a gene classification within a known database or databank, such as Gene Ontology, KEGG, RAST Subsystems, and/or Enzyme Classes. In some forms, input parameters include identifying or selecting one or more experiment parameter filter from response to specific gene knockout, bile acid, antibacterial, antibiotic, and stress. An exemplary dashboard display, including input parameter fields is set forth in FIG. 5A.

### 1. Input Commands/Query Terms

The methods examine integrated, relational datasets based on one or more user-defined inputs/query terms. Therefore, the methods include one or more steps of selecting, combining, filtering and/or calculating data in response to one or more user-defined search or query terms.

Exemplary inputs include a search parameter, for example the name of a gene, a drug, a strain or sub-species, etc. In some forms, the input is a gene classification filter.

Exemplary gene classification filters include a Rapid Annotations using Subsystems Technology (RAST) subsequence (Aziz et al., BMC Genomics. 2008; 9:75); a search field from the KEGG Database (Kanehisa, et al., Protein Sci. 31, 47-53 (2022); a Clusters of Orthologous Groups (COG) database search field (Tatusov, et al., Nucleic Acids Res. 2000 Jan. 1; 28 (1): 33-36); a gene ontology (GO) database search field (Young, et al., Genome Biology volume 11, Article number: R14 (2010)); and different enzyme classes, as well as experimental filters (i.e., omics-type of data, as well as stress and knockout profiling data). In some forms, the input includes the name of a drug or active agent, or symbol corresponding to the active agent, such as a CAS number. Exemplary active agents include therapeutic agents, such as antimicrobial drugs. Therefore, in some forms, the methods analyze the integrated relational database for interactions or changes in gene expression in a population of microorganisms in response to the presence of an antimicrobial drug.

Exemplary user-defined input includes the name of a gene, a gene function, a gene mutation, a nucleic acid sequence, the name of an organelle, a genetic pathway, a sub-species or clade, the name of a polypeptide, an amino acid sequence, a gene expression pathway, the name of a toxin, the name of a disease or disorder, the name of a virus, the name of a geographic location or place, a date, a range of dates, the name of a drug, and a host cell or organism, the name of an investigator or scientific institution, the name of a methodology; or combinations thereof. The methods include several search input types, including keywords and gene IDs. In some forms, the gene search parameters are customized by selecting a specific gene group (pangenome, core genome, metabolic core genome, essential genes, etc.), Psort B cell localization (cytoplasmic and cytoplasmic membrane), and/or gene classification (e.g., Gene Ontology, KEGG, RAST Subsystems, and Enzyme Classes). In some forms, experiment parameter filters are customized, for example, by selecting specific experiment type (e.g., response to specific gene knockout, bile acid, antibacterial, antibiotic, stress, and other treatments) and datasets (transcriptomics and/or proteomics). All of the input commands can also include one or more parameter filters. Exemplary parameter filters include, but are not limited to, setting the threshold value(s) by which a data point is included or remove from an analysis. In some forms, the methods include setting a fold-change threshold beyond which data is maintained or removed from the results. In some forms, the input specifies log2 ratio thresholds.

As demonstrated in the examples, in some forms, the input parameters include data sorted as one or more of omics type, experiment type, experiment description, locus function and [log2] threshold value.

i. Exemplary Query Terms

In exemplary forms, the query terms include: Omics type (options include transcriptomics or proteomics or both transcriptomics and proteomics); study category (options include antibacterial, antibiotic, bile acid, gene knockout, infection study, stress and treatment); specific study (options include study abbreviated by an associated publication title, investigator name, or similar); ratio (options are limited according to the specific details of a study type that is selected); Gene filters (to be selected from an external file); Keyword search for text search terms; cellular location (options include cell wall, cytoplasm, extracellular, membrane and unknown); PSORTb Localization (cell wall, cytoplasmic, extracellular, membrane, unknown); Gene set (options include pangenome, core-genome, metabolic core and essential); Enzyme class (options include hydrolases, oxidoreductases, isomerases, lyases, ligases, transferase, translocases and unclassified); COG (options include, e.g., nuclear structure, lipid metabolism, cell motility, translation, transcription, etc.); KEGG (options include, e.g., nuclear structure, lipid metabolism, cell motility, translation, transcription, etc.); RAST subsystems (options include, e.g., carbohydrates, amino acids, DNA metabolism, etc.); Gene Ontology (GO) biological processes (e.g., carbon utilization, biosynthetic processes, cell adhesion, etc.); Gene Ontology (GO) molecular function (e.g., amide binding, carbohydrate binding, cyclase activity, etc.); Gene Ontology (GO) cellular function;

UniprotKB keyword (options include, e.g., Activator, Antioxidant, Antibiotic resistance, etc.).

In some forms, the query terms each include a separate input field in a GUI display, such as a “dashboard”.

In some forms, a dashboard is divided into two or more sections, including specific functions in each section. In an exemplary form, a dashboard includes two sections, such as a left and right section as viewed by a user accessing a GUI. In exemplary forms, a first (e.g., left) section of the dashboard shows search options and user-defined parameters for the output. In an exemplary procedure, a user sets one or more search parameter (e.g., Omics type, Category of study, choice of specific studies, and ratios, etc.). The output (i.e., results) are delimited by one or more parameters, such as gene words, location of encoded proteins, the pangenome, core genome, essential genes, specific enzyme classes, COG classifications, KEGG classifications, RAST subsystems, GO biological processes, GO molecular function, UniprotKB keywords. In some forms, the dashboard includes the option to set the colors for the heatmap output, and to include hierarchical clustering. Once these parameters are set, a user initiates a search, e.g., by clicking on the button labeled “Run”.

In some forms, a second (e.g., right) section of the default landing page provides the search outputs as separate tabs. For example, in some forms, a first tab is Gene Info, which opens by default, and, prior to the initiation of searches, lists all genes in the input data and/or relational database.

In exemplary forms, the query searches C. difficile datasets, e.g., the selected source organism is C. difficile, and the input data include C. difficile-specific datasets, including data for C. difficile gene expression and/or C. difficile protein quantitation data. In an exemplary form, the genes are C. difficile genes.

In other forms, the query searches N. gonorrhoeae datasets, e.g., the selected source organism is N. gonorrhoeae, and the input data include N. gonorrhoeae-specific datasets, including data for N. gonorrhoeae gene expression and/or N. gonorrhoeae protein quantitation data. In an exemplary form, the genes are N. gonorrhoeae genes.

In yet other forms, the query searches one or more host-pathogen interaction datasets, e.g., the selected source organism is one or more pathogenic microbes and the search includes a host-interaction with the selected pathogenic microbe(s) species, and the input data include host-pathogen-specific datasets, including data for host and/or pathogen gene expression and/or protein quantitation data. In an exemplary form, the genes are human host and microbial pathogen genes.

In yet other forms, the query searches one or datasets and presents the described data connections, pathways, heatmaps and PCA using an artificial-intelligence (AI)-based analysis function.

Artificial intelligence (including natural language-based options), and tools based thereon, can be integrated into CAT-GxD and/or otherwise utilized various ways, including but limited to analyzing data, and conveying information to the user in readable text.

### E. Providing Targeted Output Data

The methods analyze the integrated relational database to provide output in the form of a targeted data set that is focused or ordered according to the user-defined input. Typically, output data are a subset of the input data and/or a subset of the integrated dataset, ordered/selected and presented to the user according to the input commands/search terms used. In some forms, the output is a subset and/or a rearrangement and/or visualization based on the pool of input data. Therefore, in some forms, the output is a filtered subset of data. Preferably, the output provides additional data based on the comparison and/or association of multiple data points within the pool of input data. For example, in some forms, the output includes a grouping of genes associated with one or more features or characteristics that was not associated with the same gene in one or more or the input datasets. In some forms, the output includes a panel of genes that were not previously associated with one another, or that were not identified as being associated with the same feature. In some forms, the output includes changes in the expression of genes in response to one or more factors, such as the presence of an antimicrobial agent or another environmental stimulus.

### 1. Presentation of Results

The methods typically provide output in the form of a subset of and/or additional data points relating to the integrated relational database. Typically, the presented data is visualized using one or more of table(s), heatmap(s), PCA(s), and protein-protein network analyses.

In some forms, the presentation of results includes visual representations of data, for example in the form of a spreadsheet, a heatmap, a list if genes, mutants, etc. Typically, the methods provide output as computer-readable code. An exemplary computer-readable code is the R statistical programming language. R provides a software environment for statistical computing and graphics. R compiles and runs on a wide variety of UNIX platforms, Windows and MacOS.

In some forms, the methods provide output as computer-readable code configured for the R shiny application. Shiny is an R package that makes it easy to build interactive web apps straight from R. In some forms, the methods provide output as computer-readable code configured for the R Studio Connect Webpage browser. See, R Shiny website, e.g., shiny.rstudio.com.

The methods can provide data using any visualization technique. In some forms, the methods present the data as one or more of a table, heatmap, and/or principal component analysis (PCA) plot. In some forms, the methods provide results in the form of a table, a heatmap, and/or a principal component analysis (PCA), e.g., within an interactive dashboard, separated by tabs. Typically, the data output table information includes log2 ratio data of genes. In some forms, when the data are presented on a computer display, the data include further interactive data, such as hyperlinks that connect to additional information, such as additional databases. In some forms, the additional databases include the NCBI gene webpage on the world wide web. Typically, heatmaps produced by the methods present gene names (y-axis) plotted against experiments (x-axis).

In an exemplary form, a user initiates an analysis of a combined SQL database formed from a total of 88 separate databases by entering the term “spore”. As depicted in FIG. 2, the methods display data in the form of a list of genes, whereby each of 181 genes are identified.

i. Interactive Display of Data

In some forms, the methods provide data in the form of an interactive interface presented on a visual display attached to a computer processor. Typically, the computer is connected to the world-wide-web and is capable of accessing one or more websites, for example, via software configured to access the world-wide-web. For example, in some forms the results include a subset of data from the pool of data in the form of a searchable and organizable database. In some forms, methods display data in the form of a general user interface (GUI), such as a “dashboard” configured with one or more input fields that enable user-defined searches and re-organization of the data. In some forms, the GUI presents the names or symbols of one or more genes or proteins on the display as an embedded hyper-link that directs the GUI, e.g., via software configured to access the world-wide-web, to one or more websites including information relating to the gene or protein and/or the source data from which the output was derived. Exemplary websites include those administered by the UniProt consortium (UniProtKB/Swiss-Prot is the expertly curated component of UniProtKB), the National Center for Bioinformatics (NCBI) and the Research Collaboratory for Structural Bioinformatics Protein Data Bank (PDB).

Exemplary forms of visual display include an interactive dashboard showing indicated genes, and/or comparing expression levels, heatmap showing expression of different genes across different samples, plots showing clusters of samples based on one or more similarities, such as a Principal Component Analysis (PCA) plot. Typically, the PCA plot does not discard any samples or characteristics (variables). Instead, it reduces overwhelming numbers of dimensions by constructing principal components (PCs). In some forms, data are presented with an option for hierarchical clustering. Genes with similar expression profiles can also be visualized via a PCA plot.

Therefore, in some forms, the methods facilitate the access and connectivity of a greater depth of information for every gene or protein identified as being relevant above a user-defined threshold to any of the user-defined search or analyses criteria than was available to the user prior to performing the search and/or analyses. In some forms, the methods enhance user access to information with one or more command or input, such as a single mouse click on a gene of interest in a GUI, such as a “dashboard” displaying data.

ii. Heatmap Display

In some forms, the methods provide data in the form of a graphical display, such as a representation of data in the form of a map or diagram in which data values are represented as colors. Therefore, in some forms, the data are presented in the form of a heat map. A heat map shows magnitude of a phenomenon, such as through the use of color in two dimensions. In an exemplary form, the variation in color may be by hue or intensity, giving visual cues to the reader about how the phenomenon is clustered or varies over space. Typically, the methods use heatmaps to demonstrate relationships between two variables, e.g., with one plotted on each axis. By cell color changes across each axis, the methods enable rapid identification of patterns in value for one or both variables. In an exemplary form, the methods render changes in gene expression as a heatmap, for example, with samples, or other variables depicted on one axis and genes depicted on the other, whereby variations in expression are indicated, for example, as variation in colors.

An exemplary system for creating and visualizing data in the form of a heatmap is the R Shiny program suite. Therefore, in some forms, the methods plot a heatmap for visualization of data using the R language with the program application R Shiny. Typically, the methods create a CSV (comma-separated values) file as a text file that has a specific format which allows data to be saved in a table structured format, for example, with cell values positive (i.e., corresponding to raw gene expression values, i.e., read counts per gene per sample) from a ChIP-seq or RNA-seq experiment. In some forms, the methods create both a static and interactive heatmap as distinct options. In other forms, the methods navigate to shinyheatmap's high performance web server (called fastheatmap) for interactive examining of datasets having larger numbers (e.g., tens or hundreds of thousands) of rows.

In some forms, the methods provide a heat map showing genes, such as the most dis-regulated genes in one or more samples. In an exemplary form, a user initiates an analysis of a combined SQL database formed from e.g., a total of 88 separate databases by entering the term “superoxide”. The methods display data in the form of a heatmap, whereby experiments are depicted on the X-axis and genes are depicted on the Y-axis. As depicted in FIG. 5C, clustering can be visually represented by the use of shading to depict similar changes in genes under different experimental conditions.

iii. Exemplary Display Options

In exemplary forms, the data visualization options include a log2 ratio threshold (0-4), a heatmap minimum color (e.g., selected from a color palate of 40 colors); a heatmap mid-color (e.g., selected from a color palate of 40 colors) and a heatmap maximum color (e.g., selected from a color palate of 40 colors), as well as option for selecting hierarchical clustering.

In some forms, the visualization options each include a separate input field in a GUI display.

a. Exemplary Expression Data Analysis Dashboard

In exemplary forms, the data visualization includes presentation on a data analysis dashboard. In an exemplary dashboard display, the user selects between display options including “Gene Info Table”, “Study Info Table”, Heatmap, Network Analysis. Pathway Analysis and Principal Component Analysis (PCA). These display options are depicted in FIGS. 5B-5E, and 6-9.

In some forms, a dashboard is divided into two or more sections, including specific functions in each section. In an exemplary form, a dashboard includes two sections, such as a left and right section as viewed by a user accessing a GUI.

In an exemplary form, a second (e.g., right) section of a dashboard provides a gene list, which upon initiation of a search, the gene list is delimited based on the output categories set by the user. The next tab (Study Info) provides a link to the original database entries, and to related publication(s), from which the data was derived. The next tab (Heatmap) provides a visual representation of the expression changes for the selected genes or groups of genes under the specified conditions. The next tab (Network) shows the interactions, if any, between the gene products as defined by STRING. The last tab displays clustering of data for each condition via Principal Component analysis.

In some forms, CAT-GxD graphical interface and interactivity includes logic filters for “Study Info” and “Gene Info”

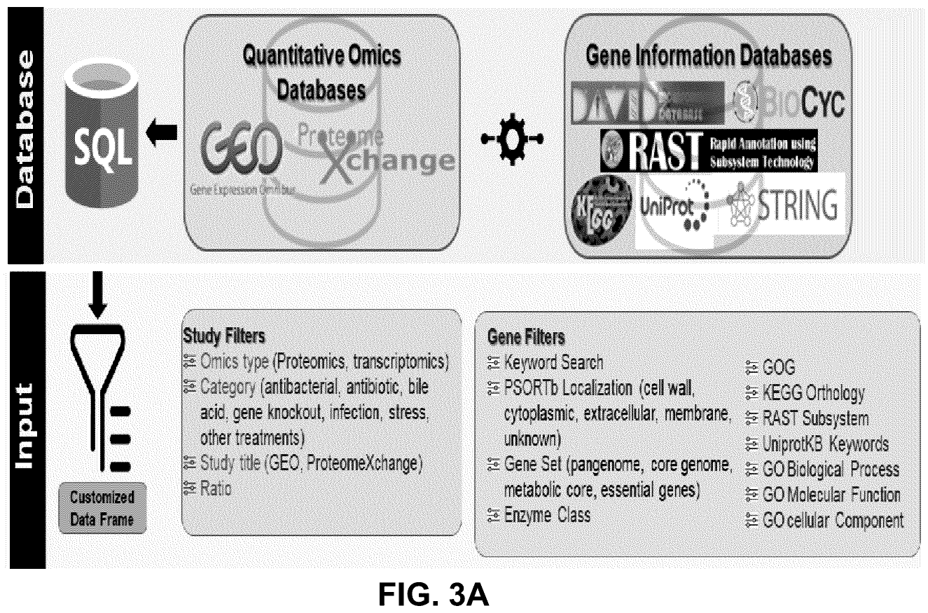

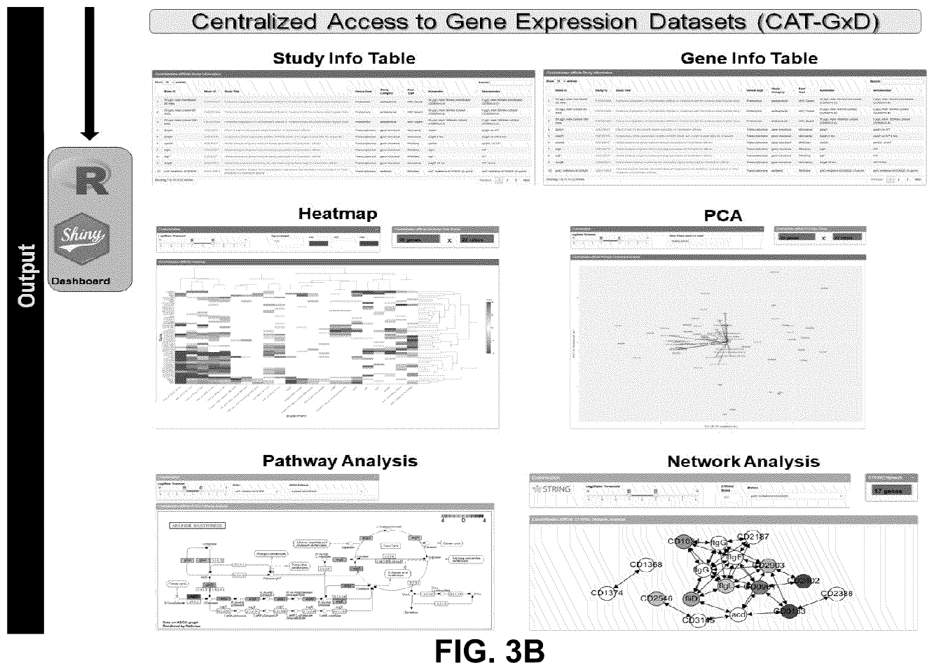

An exemplary Study Information Table and Gene Information Table generated according to the described methods includes information fields for each of Ratio ID, Study ID, Study Title, Omics Expt, Study Category, Expt Type, Numerator, and Denominator. An exemplary field data for Ratio ID is “50 microgram nlsin membrane 90 mins”. An exemplary field data for Study ID is “PXD021684”. An exemplary field data for Study Title is “Proteomic Adaptation of C. difficile to treatment with the antimicrobial peptide Nlsin”; An exemplary field data for Omics Expt is “Proteomics”. An exemplary field data for Study Category is “antibacterial”. An exemplary field data for Expt Type is “MS1 Quant”. An exemplary field data for Numerator is “50 microgram nlsin membrane 90 mins (OD 600-0.4). An exemplary field data for Denominator is “0 microgram nlsin membrane 90 mins (OD 600=0.4)”. (See, for example, FIG. 3B).

### II. Computational Systems for CAT-GxD

Disclosed are computer systems and components useful for performing, or aiding in the performance of, the disclosed CAT-GxD methods. The disclosed computer systems and components generally include combinations of articles of manufacture such as structures, machines, devices, and the like, and compositions, compounds, materials, and the like. Such combinations that are disclosed or that are apparent from the disclosure are contemplated. For example, disclosed and contemplated are systems including a computer device for accessing and processing input data, computing log2 gene expression values, compiling integrated datasets, and providing output data on a visual display.

### A. Data Structures and Computer Control

Disclosed are data structures used in, generated by, or generated from, the disclosed method. Data structures generally are any form of data, information, and/or objects collected, organized, stored, and/or embodied in a composition or medium. For example, the nucleotide sequences of a large number of samples contained within one or more databases. In some forms, the disclosed CAT-GxD method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

For example, in certain forms, the methods are implemented in computer software, or as part of a computer program that is accessed and operated using a host computer operably linked to one or more input devices, such as a keyboard, mouse, etc., and operably connected to one or more display systems, such as a monitor, printer, speaker, etc. In some forms, the methods are implemented on a computer server accessible over one or more computer networks.

FIGS. 1-4 depict an exemplary work-flow of methods that can be implemented using a computer processor operably linked to user controls, display systems and optionally accessible via one or more networks. In some forms a user accesses a computer system that is in communication with a server computer system via a network, i.e., the Internet or in some cases a private network or a local intranet. One or both of the connections to the network may be wireless. In a preferred form the server is in communication with a multitude of clients over the network, preferably a heterogeneous multitude of clients including personal computers and other computer servers as well as hand-held devices such as smartphones or tablet computers. In some forms the server computer is in communication, i.e., is able to receive an input query from or direct output results to, one or more laboratory automation systems, i.e., one or more automated laboratory systems or automation robotics that automate biochemical assays, PCR amplification, or synthesis of PCR primers. See, for example, automated systems available from Beckman Coulter.

The computer server where the methods are implemented may in principle be any computing system or architecture capable of performing the computations and storing the necessary data. The exact specifications of such a system will change with the growth and pace of technology, so the exemplary computer systems and components should not be seen as limiting. The systems will typically contain storage space, memory, one or more processors, and one or more input/output devices. It is to be appreciated that the term “processor” as used herein is intended to include any processing device, such as, for example, one that includes a CPU (central processing unit). The term “memory” as used herein is intended to include memory associated with a processor or CPU, such as, for example, RAM, ROM, etc. In addition, the term “input/output devices” or “I/O devices” as used herein is intended to include, for example, one or more input devices, e.g., keyboard, for making queries and/or inputting data to the processing unit, and/or one or more output devices, e.g., a display and/or printer, for presenting query results and/or other results associated with the processing unit. An I/O device might also be a connection to the network where queries are received from and results are directed to one or more client computers. It is also to be understood that the term “processor” may refer to more than one processing device. Other processing devices, either on a computer cluster or in a multi-processor computer server, may share the elements associated with the processing device. Accordingly, software components including instructions or code for performing the methodologies of the invention, as described herein, may be stored in one or more of the associated memory or storage devices (e.g., ROM, fixed or removable memory) and, when ready to be utilized, loaded in part or in whole into memory (e.g., into RAM) and executed by a CPU. The storage may be further utilized for storing program codes, databases of genomic sequences, etc. The storage can be any suitable form of computer storage including traditional hard-disk drives, solid-state drives, or ultrafast disk arrays. In some forms the storage includes network-attached storage that may be operatively connected to multiple similar computer servers that comprise a computing cluster.

### III. Methods of Use

The disclosed CAT-GxD systems and methods for multi-omics database analyses facilitate a broad range of data-rich applications, including identifying anti-infective targets, identifying treatment regimens, and forecasting patient outcomes.

Typically, the methods introduce user-defined analyses into multiplexed datasets of multi-omics databases relating to a microorganism, such as a pathogenic microorganism in a controllable and highly efficient manner. In some forms, the methods enable or facilitate the identification and development of treatments for diseases and disorders associated with the pathogenic microorganism in one or more host subjects.

For example, in some forms the methods analyze data associated with pathogen to identify treatment composition and/or regimens for a subject infected with or at risk of infection with the pathogen. Therefore, in some forms, the methods identify one or more therapeutic agents that are likely to provide a beneficial treatment outcome for treatment of infectious disease, and/or to provide prophylactic protection from infection by a pathogen.

In other forms, the methods identify a subject that is likely to benefit from a specific treatment regimen with one or more therapeutic agents.

In some forms, the methods additionally or alternatively facilitate the design and/or execution of additional experiments to investigate gene(s), protein(s), mechanism(s), host-pathogen interaction(s), etc., that increase the body of knowledge for the microorganism. Such information can be used to identify druggable targets of the pathogen and/or host to treat or prevent disease, and to design experiments to test the same.

### A. Correlating Microbial Genetics and Proteomics

Methods of determining the likelihood of a positive clinical outcome treatment are provided. In some forms, the methods analyze one more multi-omics databases for a pathogenic microorganism and determine the clinical outcome or predict the clinic outcome of treatment for an infection of an animal with the pathogenic microorganism.

In some forms, the determining includes providing a gene expression score for one or more genes expressed by the microorganism. For example, in some forms, the gene expression score indicates the clinical outcome of the treatment regimen. In an exemplary form, a high gene expression score compared to a median gene expression score indicates that the subject has a lower chance of a positive clinical outcome when treated with one or more therapeutic agents that are associated with the one or more genes expressed by the microorganism. In other forms, a low gene expression score compared to a median gene expression score indicates that the subject has a lower chance of a positive clinical outcome when treated with one or more therapeutic agents that are associated with the one or more genes expressed by the microorganism. In other forms, a low gene expression score compared to a median gene expression score indicates that the subject has a higher chance of a positive clinical outcome when treated with one or more therapeutic agents that are associated with the one or more genes expressed by the microorganism. In other forms, a high gene expression score compared to a median gene expression score indicates that the subject has a lower chance of a positive clinical outcome when treated with one or more therapeutic agents that are associated with the one or more genes expressed by the microorganism.

As discussed above, in some forms, the methods calculate an expression score that represents the combined expression of two or more genes, such as a signature expression score that relates to the combined changes in expression of genes in a biological pathway or cellular process that may be observed between samples. Therefore, in some forms, the methods provide a gene expression signature score for a given pathway or cellular process that can inform the susceptibility to one or more therapeutic agents. For example, a median value or score can be determined by expression of genes by a reference strain of the pathogenic microorganism. In some forms, the reference strain is an antibiotic-resistant strain of the pathogenic microorganism. In other forms, the reference strain is not known to be resistant to antibiotics.

### 1. Informing Molecular Pathways of Therapeutic Resistance

Methods for determining a likelihood of susceptibility to a specific therapeutic (e.g., antimicrobial) agent for a pathogen in a sample from a subject, based on comparison with a database of samples of including one or more of the same or different clades or strains of the same pathogen are also described. Typically, the methods identify the genomic and somatic information for each pathogen in each sample, identify correlations between one or more factors of the pathogen and susceptibility or resistance to a given treatment regimen based on data in the database for the samples as a whole, and then extrapolate the correlation(s) to provide predictions for the pathogen in the sample from the subject.

Similar strategies can be applied to other forms of organisms, for example cancer cells. The method can be used to identify correlations between one or more factors of the cancer cells and susceptibility or resistance to a given treatment regimen based on data in the database for the samples as a whole, and then extrapolate the correlation(s) to provide predictions for the cancer cells in the sample from the subject.

Typically, the methods first identify the subset of samples from the entire pool of samples sharing genomic identity with the sample from the subject, then identify a therapeutic agent, such as an antimicrobial or other therapeutic agent, to which e.g., at least 70%, 75%, 80%, 85%, 90%, 95%, or 100% of the samples in the subset are susceptible, then correlate this susceptibility with the sample from the subject to predict a positive treatment outcome for the subject if using the same agent. In this manner, the methods provide a personalized treatment regimen for one or more subjects having an infection, disease, or disorder caused by the pathogenic microorganism, cancer, etc.

In other forms, the methods identify a therapeutic agent, such as an antimicrobial or other therapeutic agent, to which less than 100%, such as 50%, 40%, 10%, or 0% of the samples in the subset are susceptible, then correlate this susceptibility with the sample from the subject to predict a corresponding treatment outcome for the subject if using the same agent. In this manner, the methods provide a likelihood of successful treatment for one or more subjects having an infection, disease or disorder caused by the pathogenic microorganism, cancer, etc.

In other forms, the methods identify a therapeutic agent, such as an antimicrobial or other therapeutic agent, to which e.g., less than 30%, 25%, 20%, 15%, 10%, 5%, or 0% of the samples in the subset are susceptible, then correlate this susceptibility with the sample from the subject to predict a negative treatment outcome for the subject if using the same antimicrobial agent. In this manner, the methods can assist in the selection of a therapeutic agent for one or more subjects having an infection, disease or disorder caused by the pathogenic microorganism, cancer, etc. for example, to avoid unnecessary use of antibiotics, to avoid expansion of antibiotic resistance, and/or to prevent unnecessary toxicity in the subject.

Therefore, in some forms, the methods identify which antimicrobial or other therapeutic agents are likely to have a positive and/or negative treatment outcome on a subject-by-subject basis.

An exemplary method for identifying one or more gene or molecular pathway in one or more microbial strain(s) responsible for resistance to a microbial agent includes:

(a) contacting a microbe of a first microbial strain with a first antimicrobial agent, wherein the first microbial strain is resistant to the first antimicrobial agent;

(b) determining a log2 gene expression ratio for one or more genes of the first microbial strain in the presence of the antimicrobial agent, wherein the determining optionally further includes evaluating gene expression and dose response data for the first antimicrobial agent; and

(c) analyzing genes that demonstrate expression and dose response correlations to identify significantly represented molecular pathways, wherein a log2 gene expression ratio greater or lower than a control value identifies genes in the first microbial strain(s) providing resistance to the first antimicrobial agent.

In some forms, the methods further include:

(d) contacting a microbe of a second bacterial strain with the first antimicrobial agent, wherein the second microbial strain is susceptible to the first antimicrobial agent;

(e) determining a log2 gene expression ratio for one or more genes of the second microbial strain in the presence of the antimicrobial agent,

wherein a log2 gene expression ratio greater or lower than a control value identify genes in the second microbial strain(s) providing susceptibility to the first antimicrobial agent.

In some forms, (d) and (e) are repeated with one or more additional “second” microbial strains (e.g., third, forth, fifth strains, etc.).

An exemplary control value is the log2 gene expression ratio of a wild type strain of the microbial strain in the absence of the antibiotic agent.

In some forms, the methods further include

(f) determining the differences in gene expression for the first and one or more second microbial bacterial strains,

wherein the differences identify molecular pathways that are responsible for susceptibility or resistance to the first antimicrobial agent.

The microbes and microbial strains can be, for example, viruses, bacteria, archaea, fungi, or protists. In preferred forms, the microbes and microbial strains are bacteria. In some forms, the antimicrobial agent is an antibiotic.

The same or similar methodology is also expressly disclosed for other organisms and disease etiologies, including but not limited to cancer. In such embodiments, the microbes of the first and second microbial strains are replaced with cancer cells of first and second cancers, and the antimicrobial agents are replaced with an alternative therapeutic agent (e.g., chemotherapy, immunotherapy, etc.). The cancers can be the same cancers from different subjects, or different cancers for the same or different subjects.

### 2. Identifying Genomic Diversity

In other forms, the methods identify genes and/or other factors that are associated with development and changes in resistance to one or more therapeutic agents among a population of pathogens or other organisms, e.g., cancer cells, for example, to guide therapeutic regimens during outbreaks of infections in one or more communities of subjects. For example, in some forms, the methods identify one or more genes or groups of genes or other factors within a sample or group of samples that are correlated with resistance of the organism (e.g., microorganisms, cancer cells, etc.) to a therapeutic agent, such as an antibiotic. Therefore, in some forms the methods identify emerging trends in the formation and development of antimicrobial or other therapy resistant genotypes and/or phenotypes. In some forms, the methods corelate the susceptibility and/or resistance of the organism (e.g., microorganisms, cancer cells, etc.) within the samples to inform the development of treatment regimens based on the likelihood of prophylactic and/or therapeutic efficacy.

### 3. Design of Effective Therapeutic and Prophylactic Agents

In other forms, the methods identify highly variable and highly conserved components of the microbial genome and/or proteome. In some forms, the identification of conserved components of a microbial genome inform the development of vaccine reagents, such as antigens and/or antigen-specific immunotherapeutic agents, such as antibodies and other immunoreceptors. In other forms, the methods identify and correlate genomic and/or proteomic changes in a subset of samples that are associated with changes in pathogenic factors, such as virulence, toxicity, infectivity, latency, likelihood of developing one or more specific symptoms or diseases, etc. Therefore, in some forms, the methods inform microbial evolution and/or divergence of one or more searchable factors.

### B. Methods of Treatment

Methods of treating a subject having or identified as being at risk of having an infection with a microbial pathogen, and/or a disease or disorder associated with an infection with a microbial pathogen, or other disease or order as discussed herein are also provided. In some forms, the methods include

(a) determining a log2 gene expression ratio for two or more genes of the microbial pathogen in the presence of a first antimicrobial agent; and

(b) administering to the subject the first antimicrobial agent in an amount effective to treat the infection if the microbial pathogen has a gene expression score for one or more genes that is equal to or less than a control value.

In some forms, the methods compile gene expression data for pathogenic strains of a microbe in the presence of one or more antimicrobial agents in a searchable database,

and treat a subject having an infection caused by a first pathogenic microorganism with the one or more antimicrobial agents when the pathogenic microorganism includes molecular pathways that are responsible for susceptibility to the first antimicrobial agent.

In other forms, the methods include not treating a subject with the one or more antimicrobial agents when the subject has an infection caused by a pathogenic microorganism including molecular pathways that are responsible for resistance to the first antimicrobial agent.

The antimicrobial agent can be, for example, an antibiotic agent, an antiviral agent, and antifungal agent, or an antiparasitic agent. See, e.g., Leekha, et al., Mayo Clin Proc. 2011 February; 86 (2): 156-167, which is specifically incorporated by reference herein in its entirety. Antibacterial agents can be bactericidal and/or bacteriostatic. Bactericidal drugs, which cause death and disruption of the bacterial cell, include drugs that primarily act on the cell wall (e.g., β-lactams), cell membrane (e.g., daptomycin), or bacterial DNA (e.g., fluoroquinolones). Bacteriostatic agents inhibit bacterial replication without killing the organism. Most bacteriostatic drugs, including sulfonamides, tetracyclines, and macrolides, act by inhibiting protein synthesis. The distinction is not absolute, and some agents that are bactericidal against certain organisms may only be bacteriostatic against others and vice versa. Although single-agent antimicrobial therapy is generally preferred, a combination of 2 or more antimicrobial agents is recommended in a few scenarios.

Non-antimicrobial therapy in the treatment of infections can include the use of operative drainage or débridement. This procedure is useful when the organism burden is very high or in the management of abscesses, for which the penetration and activity of antimicrobial agents are often inadequate.

Other therapies used in the treatment of infectious diseases involve modulating the host inflammatory response to infection. Systemic corticosteroids, thought to act by decreasing the deleterious effects of the host inflammatory response, have been found beneficial when used in conjunction with antimicrobial therapy for the treatment of bacterial meningitis, tuberculous meningitis, and Pneumocystis pneumonia in patients with AIDS. Temporary discontinuation or dose reduction of immunosuppressive agents is often required for successful treatment of infections, such as cytomegalovirus disease in organ transplant recipients or patients with rheumatologic disorders. Similarly, granulocyte colony-stimulating factor is sometimes administered to patients with prolonged neutropenia who develop invasive infections with filamentous fungi.

Intravenous immunoglobulin therapy, which acts to neutralize toxin produced by the bacteria, can be used in addition to surgical debridement and antimicrobial therapy in the treatment of necrotizing fasciitis caused by group A streptococci. Probiotics (such as Lactobacillus and Saccharomyces species) are occasionally used in the management of colitis caused by Clostridium difficile, with the hope of restoring the normal flora that has been altered by antimicrobial administration.

The same or similar methodology is also expressly disclosed for other organisms and disease etiologies, including but not limited to cancer. In such embodiments, the microbes of the first and second microbial strains are replaced with cancer cells of first and second cancers, and the antimicrobial agents are replaced with an alternative therapy, such as a therapeutic agent (e.g., chemotherapy, immunotherapy, etc.). The cancers can be the same cancers from different subjects, or different cancers for the same or different subjects.

In some embodiments, the therapy is a conventional treatment for cancer, more preferably a conventional treatment for the particular cancer type. For example, in some embodiments, the additional therapy or procedure is surgery, a radiation therapy, or chemotherapy.

In some embodiments, the conventional cancer therapy is in the form of one or more active agents. Therefore, in some embodiments, the methods administer compositions in combination with one or more additional active agents. Such active agent can be, for example, chemotherapeutic agents, cytokines, chemokines, radiation therapy, or immunotherapy. The majority of chemotherapeutic drugs can be divided into alkylating agents, antimetabolites, anthracyclines, plant alkaloids, topoisomerase inhibitors, and other antitumor agents. These drugs affect cell division or DNA synthesis and function in some way. Therapeutics include monoclonal antibodies and the tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors).

In some embodiments, the therapy is a chemotherapeutic agent. Representative chemotherapeutic agents include, but are not limited to, amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ (2) 5, and combinations thereof.

In some embodiments, the treatment is or includes immunotherapy such as inhibition of checkpoint proteins such as components of the PD-1/PD-L1 axis or CD28-CTLA-4 axis using one or more immune checkpoint modulators (e.g., PD-1 antagonists, PD-1 ligand antagonists, and CTLA4 antagonists), adoptive T cell therapy, and/or a cancer vaccine. Exemplary immune checkpoint modulators used in immunotherapy include Pembrolizumab (anti-PD1 mAb), Durvalumab (anti-PDL1 mAb), PDR001 (anti-PD1 mAb), Atezolizumab (anti-PDL1 mAb), Nivolumab (anti-PD1 mAb), Tremelimumab (anti-CTLA4 mAb), Avelumab (anti-PDL1 mAb), and RG7876 (CD40 agonist mAb).

In some embodiments, the treatment is or includes adoptive T cell therapy. Methods of adoptive T cell therapy are known in the art and used in clinical practice. Generally adoptive T cell therapy involves the isolation and ex vivo expansion of tumor-specific T cells to achieve greater number of anti-tumor T cells than what could be obtained by vaccination alone. The tumor-specific T cells are then infused into patients with cancer in an attempt to give their immune system the ability to overwhelm remaining tumor via T cells, which can attack and kill the cancer. Several forms of adoptive T cell therapy can be used for cancer treatment including, but not limited to, culturing tumor infiltrating lymphocytes or TIL; isolating and expanding one particular T cell or clone; and using T cells that have been engineered to recognize and attack tumors.

In some embodiments, the T cells are taken directly from the patient's blood. Methods of priming and activating T cells in vitro for adaptive T cell cancer therapy are known in the art. See, for example, Wang, et al, Blood, 109 (11): 4865-4872 (2007) and Hervas-Stubbs, et al, J. Immunol., 189 (7): 3299-310 (2012).

In some embodiments, the treatment is or includes a cancer vaccine. Vaccination typically includes administering a subject an antigen (e.g., a cancer antigen) together with an adjuvant to elicit therapeutic T cells in vivo. In some embodiments, the cancer vaccine is a dendritic cell cancer vaccine in which the antigen is delivered by dendritic cells primed ex vivo to present the cancer antigen. Examples include PROVENGE® (sipuleucel-T), which is a dendritic cell-based vaccine for the treatment of prostate cancer (Ledford, et al., Nature, 519, 17-18 (5 Mar. 2015). Such vaccines and other compositions and methods for immunotherapy are reviewed in Palucka, et al., Nature Reviews Cancer, 12, 265-277 (April 2012).

In some embodiments, the compositions and methods are used prior to or in conjunction with surgical removal of tumors, for example, in preventing primary tumor metastasis. In some embodiments, the compositions and methods are used to enhance the body's own anti-tumor immune functions.

The disclosed systems and methods allow for making informed decisions on treatment strategies. For example, in some forms, identification of strain or cells as being resistant to an antibiotic(s) or other therapy and will be allow the practitioner to deselect antibiotic(s) or other therapies to which the microbes or other organism (e.g., cancer cells, etc.) are resistant and/or select an antibiotic or other therapy to which the microbes or other organism are not resistant.

The disclosed compositions and methods can be further understood through the following numbered paragraphs.

1. A method for analysis of the homology and/or differential expression of one or more user-defined genes and/or gene functions from a pool of genomic sequence data derived from a multiplicity of samples of organisms of the same species, the method including

(i) determining homology between the multiplicity of samples to identify common genes;

(ii) identifying differential expression of the common genes; and

(iii) presenting data reporting the homology and/or differential expression of the user-defined genes and/or gene functions.

2. The method of paragraph 1, wherein the presenting in step (iii) includes identifying relationships between one or more genes within samples of the multiplicity of samples.

3. The method of paragraph 1 or 2, wherein the methods are implemented on a computer, and wherein the pool of genomic sequence data is provided in the form of a computer-readable database.

4. The method of paragraph 3, wherein the computer-readable database includes one or more of gene expression data, transcriptomics data, protein abundance data, and proteomics data.

5. The method of paragraph 4, wherein the databases include the Gene Expression Omnibus (GEO) database and/or the ProteomeXchange database, the Gene Ontology (GO) database, KEGG (Krypto Encyclopedia of Genes and Genomics) database, RAST Subsystems database (Clusters of Orthologous Genes), and Enzyme Classes.

6. The method of paragraph 4 or 5, wherein steps (i) and/or (ii) include one or more of:

(a) determining sequence homology between two or more of the multiplicity of samples of the organism;

(b) determining differential gene expression and/or abundance, including log2 ratio, median normalization and one-sample t-test between two or more of the multiplicity of samples of the organism;

(c) principal component analysis (PCA); and

(d) data structuring, including column identity and position for data points.

7. The method of any one of paragraphs 1-6, wherein steps (i) and/or (ii) include the creating, storing, updating and/or retrieving of data including quantitative proteomic analysis and/or transcriptomic analysis two or more as a relational database using structured query language (SQL).

8. The method of any one of paragraphs 3 to 7, wherein presenting data in step

(iii) is implemented through one or more computer programs including RShiny, RStudio Connect, gene set enrichment analysis (GSEA).

9. The method of paragraph 8, wherein visualization includes a heat map representation of changes in the expression of one or more genes amongst two or more of the multiplicity of samples.

10. The method of any one of paragraphs 3-9, wherein the analysis is initiated by one or more user-defined input terms entered through a user interface connected with the computer.

11. The method of paragraph 10, wherein the user-defined input is selected from the group including the name of a gene, a gene function, a gene mutation, a nucleic acid sequence, the name of an organelle, a genetic pathway, a sub-species or clade, the name of a polypeptide, an amino acid sequence, a gene expression pathway, the name of a toxin, the name of a disease or disorder, the name of a virus, the name of a geographic location or place, a date, a range of dates, the name of a drug, and a host cell or organism, the name of an investigator or scientific institution, a type/classification of a study, a gene expression log2 ratio, a keyword search term, an enzyme class, gene grouping, and the name of a methodology; or combinations thereof.

12. The method of paragraph 10, wherein the user-defined input is the name of a gene or a code corresponding to a gene, and

wherein the presenting in step (iii) includes identifying one or more polymorphisms within the gene in the pool of genomic sequence data,

optionally wherein the presenting also identifies the samples associated with each polymorphism.

13. The method of any one of paragraphs 10 to 12, wherein the user-defined input term is the name of a gene or a code corresponding to a gene, and wherein the presenting in step (iii) includes differential expression of the gene amongst the pool of genomic sequence data.

14. The method of any one of paragraphs 1-13, wherein input parameters include selecting a gene in one or more from the group including pangenome, core genome, metabolic core genome, and essential genes.

15. The method of any one of paragraphs 1-14, wherein input parameters include selecting a gene from the cell localization cytoplasm or from cytoplasmic membrane.

16. The method of any one of paragraphs 1-15, wherein input parameters include selecting an experiment parameter filter from the group including response to specific gene knockout, bile acid, antibacterial, antibiotic, and stress.

17. The method of any one of paragraphs 1-15, wherein input parameters include selecting a search term from a database selected from the group including KEGG (Krypto Encyclopedia of Genes and Genomics), GO (Gene Ontology), COG (Clusters of Orthologous Genes), RAST (Rapid Annotations using Subsystems Technology) and UNIPROTKB.

18. The method of any one of paragraphs 1-16, wherein the organism is a microorganism selected from the group including a bacteria, a virus, a fungi, a protozoan, an alage and an archaebacteria.

19. The method of paragraphs 18, wherein the microorganism is a bacteria.

20. The method of paragraph 18, wherein the microorganism is a pathogenic microorganism associated with one or more diseases or disorders in humans.

21. The method of paragraph 6, further including

(iv) determining or correlating gene expression of one or more genes expressed by the organism that is known to be associated with resistance or susceptibility to one or more active agents.

22. The method of paragraph 21, wherein an increase in gene expression compared to a reference or median gene expression informs the organism has an increased chance of survival in the presence of the one or more active agents.

23. The method of paragraph 21 or 22, further including

(v) correlating treatment options for an infection of a subject with the organism(s) with changes in gene expression to determine clinical outcome.

24. The method of paragraph 23, wherein an increase in gene expression compared to a reference or median gene expression indicates that the subject has a lower chance of a positive clinical outcome when treated with one or more therapeutic agents that are associated with the one or more genes expressed by the organism.

25. The method of paragraph 24, wherein the positive clinical outcome is therapeutic efficacy of the therapeutic agent.

26. The method of paragraph 24, wherein the positive clinical outcome is survival of the subject.

27. The method of any one of paragraphs 22-26, wherein the gene expression value is determined by calculating a log2 gene expression value, optionally wherein a positive log2 fold change corresponds to increased expression, whereas negative values correspond to decreased expression.

28. The method of any one of paragraphs 22-27, wherein the median or reference value is determined by expression of genes of a reference or control dataset.

29. The method of any one of paragraphs 24-28, further including

(vi) treating a subject in need thereof for an infection with the organism by administering to the subject the therapeutic agent in an amount effective to treat the infection if the organism has a negative log2 fold change.

30. A method for identifying molecular pathways in one or more pathogenic microbial strain(s) in response to an active agent, including

(a) contacting a microbe of a first microbial strain with a first active agent;

(b) determining a change in gene expression for one or more genes of the first microbial strain in the presence of the active agent, wherein the determining optionally further includes evaluating gene expression and dose response data for the first active agent; and

(c) analyzing genes that demonstrate expression and dose response correlations to identify significantly represented molecular pathways, wherein a change in gene expression greater or lower than a reference or median value identifies genes in the first microbial strain(s) responsive to the first active agent.

31. The method of paragraph 30, wherein the control value is the gene expression of a wild type strain of the microbial strain in the absence of the active agent.

32. The method of paragraph 30 or 31, further including

(d) determining differences in gene expression between the first and a second or further microbial strain in the presence of the same active agent, wherein the differences identify molecular pathways that are responsible for different phenotypic and/or genotypic responses to the first active agent.

33. The method of paragraph 32, further including

(e) compiling the gene expression data for the first and second or further microbial strains in the presence of the first active agent in a database, wherein the data base is searchable.

34. The method of paragraph 33, wherein the database further includes gene expression data for a multiplicity of different microbial strains, and/or a multiplicity of different active agents.

35. The method of paragraph 34, wherein the first active agent is an antimicrobial agent, and wherein the phenotypic and/or genotypic responses to the first active agent include susceptibility or resistance to the antimicrobial agent.

36. The method of paragraph 35, further including treating a subject having an infection caused by a pathogenic microorganism with the first active agent when the pathogenic microorganism includes molecular pathways that are associated with susceptibility to the first active agent,

wherein the molecular pathways of the pathogenic microorganism are determined by comparing gene expression data of the pathogenic microorganism to those in the database.

37. The method of paragraph 36, further including not treating a subject with the first active agent when the subject has an infection caused by a pathogenic microorganism including molecular pathways that are responsible for resistance to the first antimicrobial agent,
wherein the molecular pathways of the pathogenic microorganism are determined by comparing gene expression data of the pathogenic microorganism to those in the database.

38. The method of any one of paragraphs 1-29, wherein the organism is a pathogenic microorganism/microbial strain

39. The method of any one of paragraphs 30-38, wherein the pathogenic microorganism/microbial strain is selected from the group including bacteria, viruses, archaea, fungi, and protists.

40. The method of any one of paragraphs 30-39 wherein the microorganism/microbial strain is selected from the group including Clostridioides spp., Neisseria spp., Candida spp., Enterobacteriaceae spp., Acinetobacter spp., Campylobacter spp., and Escherichia spp.

41. The method of any one of paragraphs 30-40, wherein the pathogenic microorganism/microbial strain is selected from the group including Clostridioides difficile, Neisseria gonorrhoeae, Candida auris, and Escherichia coli.

42. The method of paragraph 30-41, wherein the microorganism/microbial strain is an antibiotic-resistant bacterium selected from a carbapenem-resistant Enterobacteriales sp. and a carbapenem-resistant Acinetobacter sp.

43. The method of any one of paragraphs 20-42, wherein the results are implemented on a computer display in the form of an interactive user interface.

44. The method of paragraph 43, wherein the output is in the form of a principal component analysis (PCA) for gene expression and/or a heatmap.

45. The method of paragraph 44, wherein the heatmap includes embedded hyperlinks for each of the genes within the heatmap.

### EXAMPLES

### Example 1: Establishment of a Platform for Streamlined Access and Analysis of Multi-Omic Bacterial Gene Expression Data

Initially focusing on the Centers for Disease Control (CDC) Urgent Threat pathogen Clostridioides difficile, CAT-GxD (Centralized Access to Gene Expression Datasets), was developed as an integrated and queryable engine to readily access and compare expression of specific genes or gene subsets in accordance with the Findability, Accessibility, Interoperability, and Reusability (FAIR) guiding principles (6). By providing integrated access to disparate datasets and embedded tools for comparative analysis and discovery, CAT-GxD provides insights into bacterial physiology and pathogenesis.

### Design Concept

A goal of the CAT-GxD initiative is to address pressing infection/immunity challenges by developing targeted solutions under a bedside-to-bench-to-bedside paradigm. Specifically, CAT-GxD is an integrated, queryable pathogen/host altas of CDC “Urgent Threat” Pathogens. CAT-GxD enables active and iterative screening of data from the broader UA community as well as federal and foundation funding agencies and industry.

The scope and possibilities of informatics-driven bio-solutions are still meager compared to the scale of the global infectious disease burden. The lack of targeted (e.g., microbiota-sparing) anti-infective strategies over the past decade represents a huge unmet medical need. There is tangible potential to launch an ambitious and long-lasting venture to improve the lives of humans and animals via integration of multi-omics datasets to reveal anti-infective targets. CAT-GxD is thus premised on establishing a University-wide, infection-immunity-focused, and data-driven resource.

CAT-GxD seeks to identify market-responsive anti-infective targets that will address infectious disease challenges under a One Health paradigm.

There have been multiple high-profile failures in clinical trials focused on vaccines and anti-infectives (Sanofi C. difficile vaccine 2018, and Pfizer, C. difficile vaccine 2022). Industry experts require input from academic research centers for effective strategies for mitigating the impact of such losses in investment; to access anti-infective-relevant datasets as they are generated worldwide; and to participate early in the R&D process by engaging with scientists who identify effective anti-infective targets from CAT-GxD. Ultimately, it is a tool that facilitates real-world, and immediate, informatics-driven identification of infectious disease interventions, using an integrated and query able platform to extract information on specific genes or gene subsets for CDC Urgent Threat bacterial pathogens. A schematic representation of the CAT-GxD concept is presented in FIG. 1. CAT-GxD is premised on a platform that integrates datasets from diverse sources, pathogen variants and multi-omics approaches to facilitate generation of scientific hypotheses and identify anti-infective targets.

Publicly available gene expression compendia include transcriptomics (RNAseq and microarray) and proteomics datasets, as well as regulatory and structural information. For bacteria, such expression data are available for diverse isolates, mutant derivatives, and upon exposure to varied environmental conditions. While the datasets can be retrieved from Gene Expression Omnibus (GEO) database (1, 2) and ProteomeXchange Consortium (3-5), these data are not readily accessible or queryable for reasons including the use of non-standardized gene IDs across multiple datasets, presence of replicate ratios assigned to a gene, and availability of only the raw dataset without log2 ratio calculations to compare samples.

### Methods

### Model Organism Selection

CAT-GxD is developed using Clostridioides difficile as a model organism, with the platform being adaptable for other bacteria as well as bacterium/host datasets.

The bacterial pathogen Clostridioides difficile, the most common cause of healthcare-associated bacterial infections in the US, is considered an Urgent Threat to US healthcare by the CDC. Annual cases range from 500,000-1,000,000 resulting in ˜29,000 deaths and >$6.3 billion cost overall to healthcare systems. There are currently no vaccines against C. difficile infections, and there are several limitations to antibiotic therapy, including the emergence of resistant strains. The mechanisms by which C. difficile causes disease are poorly understood and are an active area of investigation worldwide. The ˜4,000 genes of C. difficile are controlled by complex regulatory networks that are responsive to metabolic and environmental cues; the functions of most of these genes in virulence remain undefined.

### Implementation

The CAT-GxD engine was developed on a R shiny (version 1.7.5) (18) framework that utilizes the dplyr (version 1.1.4) (19) for the data filtering, cyjShiny (version 1.0.42) (20) for interactive networks, heatmaply (version 1.4.2) (21) for interactive heatmap, ggbiplot (version 3.4.4) (22) for principal component analysis (PCA) visualization, and Pathview (version 3.18) (23) and KEGGREST (version 1.42.0) (24) for KEGG pathway analysis. The publicly accessible engine is published using the RStudio Connect platform, and is freely available on the world-wide web address at viz.datascience.arizona.edu/catgxd/. CAT-GxD was validated on multiple web browsers including Mozilla Firefox, Google Chrome, Microsoft Edge, and Safari.

### Omics Resources

Typically, specific gene expression datasets and protein quantitation datasets for the selected target organism are collected and made available. In an exemplary form, C. difficile-specific gene expression datasets and protein quantitation datasets were collected from GEO database and ProteomeXchange databases, respectively. An SQL database was generated using RSQlite (version 2.3.4) (25) to incorporate the 56 nonredundant GEO transcriptomics datasets and 8 quantitative proteomic datasets. GEO subseries datasets already available in their respective superseries dataset were excluded. Quantitative total proteomic datasets were included; those focused on post-translational modification studies were excluded. To integrate and normalize the datasets, gene IDs were matched to the Ensembl stable IDs of the widely accepted C. difficile 630 reference strain using BLASTp, using protein identity and gene position comparisons and given the NCBI format CD630. Pre-computed log2 ratios without any gene duplication were imported as is. Studies with replicate log2 ratios assigned for a gene ID (common in microarray experiments due to replicate spots) were solved by computing the median log2 ratio. Two-channel microarray experiments with duplicate ratios assigned to a gene were solved by computing the average of the complementary ratios then log2 transformed. Reads Per Kilobase Million (RPKM) of raw RNASeq data were computed. For datasets without any computed ratios, log2 ratios were calculated for each treatment vs. control, and median normalization was performed. Gene classifications (described in Input and Data Filtering) were compiled to provide a comprehensive and robust search.

### Accessibility

CAT-GxD provides easy access to a large set of multi-omics gene expression datasets generated from multiple strains of the selected organism. For example, in some forms, CAT-GxD provides access to multi-omics gene expression datasets generated from multiple C. difficile strains and mutant derivatives, and captures gene expression changes in response to various environmental and growth conditions. A user interface was carefully designed to allow researchers to easily navigate the various CAT-GxD analysis tools. Popup windows are available to orient users on how to customize the different input and search parameters. An integrated tutorial video demonstrates how to use and navigate the CAT-GxD platform. A Demo setting with preset filters is included to orient the first-time user.

Input parameters and Data filtering

CAT-GxD accepts several search input types, including keywords and gene IDs (FIG. 1). In the Data Frame tab, gene search parameters can be customized by selecting specific PSORTb (v3.0.3) protein localization (cell wall, cytoplasmic, extracellular and membrane), gene set (pangenome, core genome, metabolic core genome, and essential genes), and functional classification categories (enzyme class, clusters of orthologous genes (COG), Kyoto Encyclopedia of Genes and Genomes (KEGG) Orthology, Rapid Annotation using Subsystem Technology (RAST)

Subsystems, UniProtKB keywords, Gene Ontology (GO) biological process, GO molecular function, and GO Cellular Component). Experiment parameter filters can be customized by selecting specific omics type (proteomics and/or transcriptomic), category (e.g., response to specific antibacterial, antibiotic, bile acid, gene knockout, infection, stress, and other treatments), specific experiment study and specific sample comparisons or ratios. Users can also specify log2 ratio (“stringency”) thresholds.

In some forms, the input parameters include selecting a set of genes that are filtered according to the specific organism that is the subject of the search. For example, in some forms, the input parameters include selecting a gene filter that is specific to C. difficile, or to a different organism for which a database exists, such as Neisseria gonorrhoeae.

### Visualization

CAT-GxD presents the data as a gene information table, study information table, heatmap, principal component analysis (PCA) plot, KEGG pathway analysis maps, and STRING network analysis, separated by tabs. As an example, output from CAT-GxD presents data as a table, heatmap, and principal component analysis (PCA) plot, separated by tabs (FIGS. 5A-5E). Output table information include log2 ratio data of genes. Genes are hyperlinked to NCBI gene page. Heatmap presents gene names (y-axis) plotted against experiments (x-axis). An option for hierarchical clustering is provided. Genes with similar expression profiles can also be easily visualized via the PCA plot.

### Gene Information Table

The gene information table provides gene ID, gene description, isoelectric point (pI), molecular weight (MW), amino acid length, and PSORTb localization. Gene IDs are hyperlinked to AlphaFold Protein Structure Database pages. Gene descriptions are hyperlinked to NCBI Pubmed pages with pre-populated search words (gene name, gene ID, PMID) to aid in locating published papers describing the gene. Gene or operon coordinates are hyperlinked to National Center for Biotechnology (NCBI) Sequence Viewer 3.49.0 pages.

### Study Information Table

The study information table contains hyperlinks to specific GEO series accession number (GSExxx), ProteomeXchange dataset identifier (PXD+six figure integer), and publication associated with each experiment ID. Specific samples compared are also listed.

### Heatmap

An interactive, hierarchically clustered heatmap presents expression ratios for each gene (y-axis) plotted against selected experiment IDs (x-axis). Log2 ratio thresholds and heat map colors can be customized. Gene name, experiment ID and actual log2 ratio value can be visualized by hovering the cursor over a specific heatmap cell. Users can narrow the set of genes and experiments in view by dragging a rectangle around a desired region of the heatmap. Zoom and pan functions are also available via icons/buttons on the right, and above the heatmap. Increasing the figure height allows for separation of text that may be clustered on the X axis.

### Principal Component Analysis (PCA).

Genes with similar expression profiles can also be easily visualized via the interactive PCA plot. If desired, PCA plot can also be zoomed in by dragging a rectangle around a region, or by using the buttons on the right, above the PCA plot.

### STRING Network Analysis

For each of the selected ratio ID, log2 ratios are overlaid on Search Tool for the Retrieval of Interacting Genes/Proteins (STRING) networks. STRING score thresholds can be customized. Users can also reposition nodes in the network. Output network is hyperlinked to STRING Database page. Clicking on STRING will transfer users to the STRING site where the nature of interactions between the proteins can be gleaned (but the CAT-GxD expression data will not carry over). The STRING database website will pop up in a new window. STRING has the option to add more nodes to the inputted genes by pressing the ‘More” button. Nodes can be added multiple times in order to connect some or all of the identified networks. In some forms, one or more cluster may be unable to connect to the main network after one or more cycles of adding nodes. The least number of nodes with the higher network association scores is selected to bridge the clusters; nodes are identified to connect the separate clusters. The bridging nodes are added to and re-entered in CAT-GXD's ‘Search’ bar to connect to the network.

### Kyoto Encyclopedia of Genes and Genomes (KEGG) Pathway Analysis.

CAT-GxD's pathway analysis gives the user the capability of overlaying gene expression ratios on the selected KEGG pathway. For each of the selected ratio ID, log2 ratios are overlaid on selected KEGG analysis pathway maps. Log2 ratio threshold can be customized. This feature can be used to further investigate the differences in the gene expression of each gene cluster identified.

### Results

In the exemplary method, quantitative omics datasets for CDC Urgent Threat pathogen C. difficile available from GEO and ProteomeXchange databases were curated, along with gene information from Gene ontology, KEGG, COG, RAST and STRING databases, to build an integrated database.

The exemplary method demonstrated that users can customize queries for their specific gene(s) of interest by selecting gene classification filters, experimental filters, and setting the fold change threshold. Gene expression data is visualized in the interactive dashboard as heatmaps, PCA plots and Gene Info Table.

There are 2 other databases that attempt to integrate expression data for C. difficile. Exemplary other databases include the Bacterial and Viral Bioinformatics Resource Center (available online at the world-wide web address “bv-brc.org”); the Cdiff Portal (available online at the world-wide web address “networks.systemsbiology.net/cdiff-portal/”); and the MicrobesOnline database (available online at the world-wide web address “microbesonline.org/”).

When compared against the CAT-GxD system coverage, using “sporulation genes” as an example, the other databases identified a total of 79 and 58 genes, respectively, whereas CAT-GxD identified 181, including all but 2 of the genes found in the other two databases.

These data are set forth in FIG. 2 (numbers within each circle represent genes that are “found” by each database when the query “spore” is entered).

A queryable spreadsheet incorporating over 30 datasets was generated for the healthcare-acquired pathogen, Clostridioides difficile (FIGS. 3A-3B); VBA Macros are used for querying, filtering, and exporting of data. For this work, data wrangling was used to match various data formats to gene IDs of a pangenome from different C. difficile strains. Representative log2 ratios of each data series were computed, normalized, and tested for significant change. Data was then restructured into a format that could be read by the R Bioconductor package for subsequent visualization, and analyses, such as gene set enrichment analysis (GSEA). Therefore, data wrangling is a step that is incorporated into a pipeline for faster updating of the database.

As set forth in FIG. 3A, quantitative omics datasets for CDC Urgent Threat pathogen C. difficile available from GEO and ProteomeXchange databases were curated and integrated, along with gene information from Gene ontology, KEGG, COG, RAST and STRING databases (1-5, 30-37, 39, 40), to build CAT-GxD search engin; users can select preferred experimental and gene filters to create a customized data frame. As depicted in FIG. 3B, gene expression data can be visualized as gene information table, study information table, heatmaps, PCA plots, STRING network analysis and KEGG pathway maps.

Using the workflow established above, the system provides an SQL database structured in such a way that queries can be made to both the gene list and to gene expression studies, as well as a world-wide-web interface that is used to retrieve data based on the keyword input and log2 ratio/p-value cutoff settings. An SQL dashboard provides for quick visualization of retrieved data as user-friendly “gene cards” as an output. A flow chart depicting an exemplary sequence of data wrangling and web interphase for the SQL database is set forth in FIG. 4. A visual representation of the Dashboard is provided in FIGS. 5A-5E, and an exemplary presentation of data in the format of a “gene card” is set forth in FIG. 5B.

Exemplary Use Case Test study

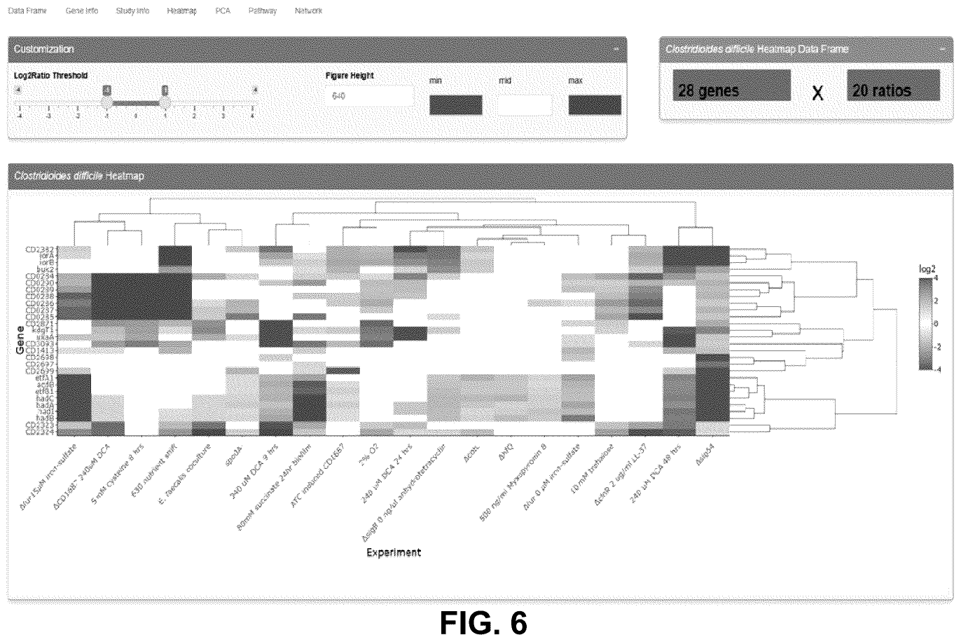

Use case: Application of Centralized Access to Gene Expression Datasets is demonstrated by re-analyzing previously published data on the contribution of SigL to C. difficile biology. SigL, also known as rpoN (RNA polymerase, nitrogen-limitation N) and sig54 (Sigma-54), is an alternative sigma factor that facilitates bacterial adaptation to the environment. SigL-binding promoter sites, and SigL-dependent transcriptomic and proteomic changes in C. difficile, have been characterized by several groups. The following use-case example includes an overview of input and output data, including the visualization/representation of datasets using each of a heatmap representation, principal component analysis and STRING pathway analysis (depicted in FIGS. 6-9).

### Use Case: Overview

CAT-GxD aims to serve as a gateway to gene expression and protein abundance datasets. Researchers can uncover new insights and forge connections that may have remained obscured in the sheer volume of existing data. This tool not only streamlines the investigative process but also amplifies the potential for discovery in the field of genomics and proteomics. The value of CAT-GxD can be demonstrated by re-analyzing a laboratory's previously published data on exploring the contribution of SigL to C. difficile biology. SigL, also known as rpoN (RNA polymerase, nitrogen-limitation N) and sig54 (Sigma-54), is an alternative sigma factor that facilitates bacterial adaptation to the extracellular environment. The C. difficile genome was searched for promoter sites based on the C. difficile sigL PWM (position weight matrix) using FIMO (Find Individual Motif Occurrences). Identified sigL sites with FIMO q-values <0.05 located <200 bp upstream of the gene and/or operon were compiled, tabulated, and cross-referenced to previously identified C. difficile sigL promoters and sigL::erm microarray results. Using the microarray dataset as the reference ratio (Δsig54), it was observed that of the 16 genes and 15 operons identified to have an upstream sigL promoter site, only 4 genes and 4 operons showed reduced expression patterns in the sigL deletion mutant.

There does not appear to be any correlation between higher identity scores to the motif and reduced expression.

### Use Case: Data

Using the Δsig54 microarray dataset as the reference ratio, it was observed that of the 16 genes and 15 operons with predicted SigL promoter sites, only 4 genes and 4 operons (totaling 28 genes) had reduced expression in Δsig54 relative to the parent strain. With an input of these 28 genes in the ‘Gene Filters’ search bar in the CAT-GxD Data Frame tab, 123 of 144 ratios had gene expression or protein abundance ratios greater or less than the log2 ratio cutoff of +1 or −1.

### Use Case: Heatmap

Twenty ratios, including the reference Δsig54 ratio, were selected in the ‘Study Filters’ menu in the Data Frame tab to reduce the size and complexity of the heatmap. The Heatmap tab highlights ratios that had similar and dissimilar expression patterns relative to Δsig54 (See FIG. 6). The 28 downregulated genes were inputted into the search bar to generate a heatmap. 123 of the 144 ratios had gene expression or protein abundance ratios greater or less than the log2 ratio cutoff of +1 or −1. 20 ratios, including the reference ratio of sig54, were selected in ‘Data Frame’ tab to reduce the size and complexity of the heatmap highlighting ratios that have similar and dissimilar expression patterns to sig54.

### Use Case: Principal Component Analysis

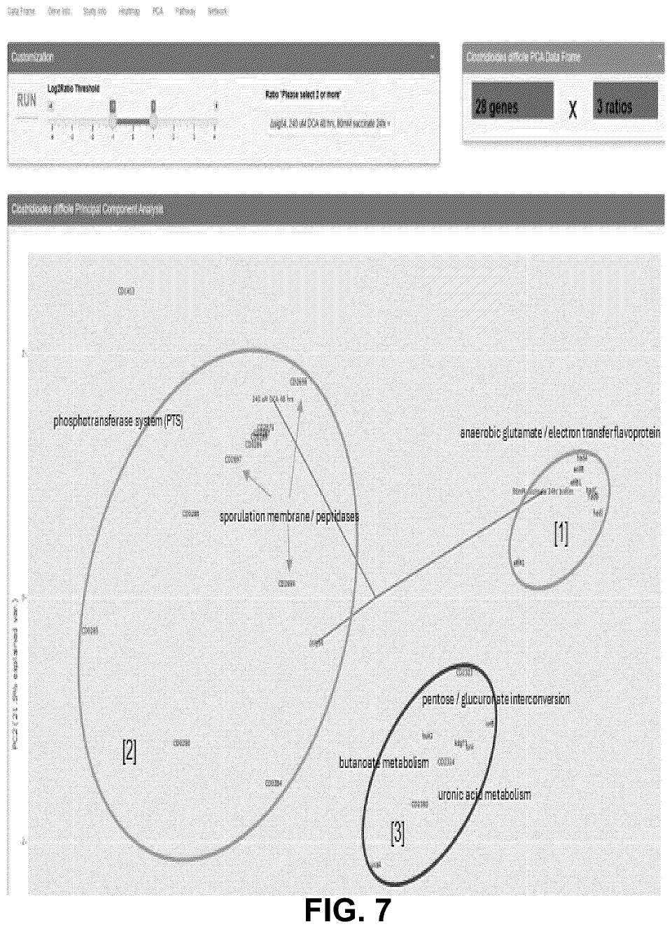

Three ratios, the reference (Δsig54), the most similar (240 μM DCA 48 hrs), and the most dissimilar (80 μM succinate 24 hrs biofilm), were selected for more detailed analysis. Both DCA and succinate induce biofilm formation in C. difficile with the latter inducing a significantly thicker biofilm. As depicted in FIG. 7, when these three ratios were selected under the PCA (Principal Component Analysis) tab, 3 gene clusters with similar expression patterns could be discerned:

1. An anaerobic glutamate/electron transfer flavoprotein cluster (hadA . . . 5 . . . etfA1 [467033 . . . 474494]),

2. A phosphotransferase system (PTS) cluster (CD0284 . . . 5 . . . . CD0290 [346714 . . . 350720]) and sporulation membrane/peptidases (CD2697 . . . 1 . . . CD2699 [3117803 . . . 3118351]), and

3. A butanoate metabolism (buk . . . 2 . . . . CD2382 [2745688 . . . 2748166]) and pentose/glucuronate interconversion (CD2323. . . . CD2324 [2686241 . . . 2686241]) and uronic acid metabolism (kdgT1 . . . 1 . . . uxaA [3357631 . . . 3360077]) cluster.

These 3 clusters represented genes/operons with similar expression patterns based on the 3 selected ratios.

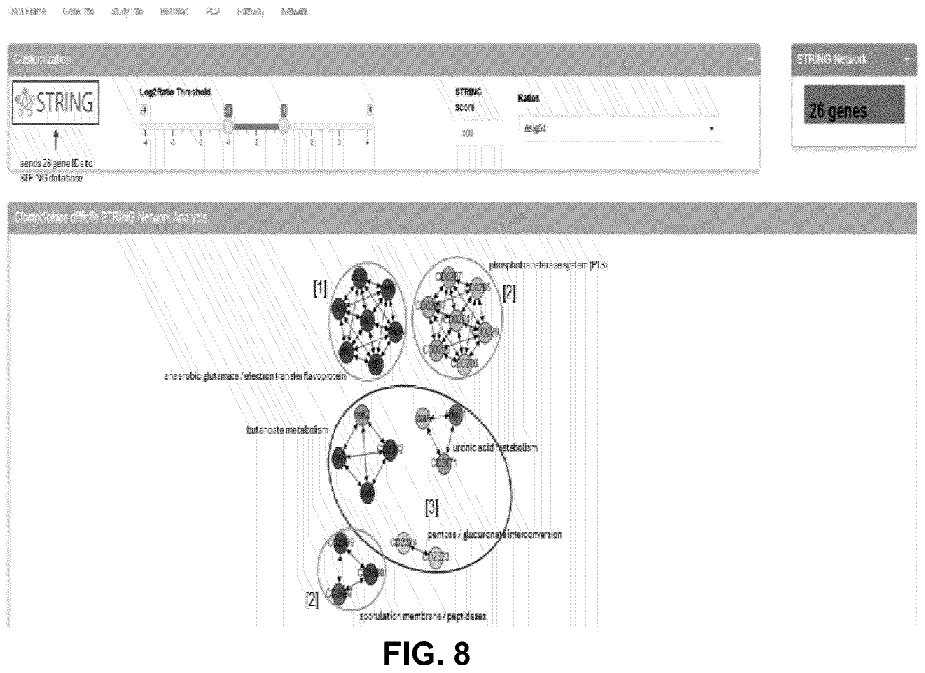

The genes downregulated in Δsig54, relative to WT, can next be viewed as a network of interactions defined by the STRING database. The 28 genes resolved into six networks; CD1413 and CD3093 did not have interactions within the selected gene set, and were excluded from further analysis. To glean additional insight into the interactions between the remaining 26 genes, the analysis was transitioned to the STRING database by clicking on the STRING icon at the top left of the page (expression information, however, will not ‘carry over’). Clicking the ‘More’ button in STRING provides the option of adding more nodes to the selected genes; thus, it was possible to add nodes 7 times to connect 5 of the 6 networks. After more than 10 cycles of adding nodes, however, the 3 gene sporulation membrane/peptidases (CD2697 . . . 1 . . . CD2699 [3117803 . . . 3118351]) cluster did not connect to the main network. The least number of nodes with the higher network association scores were selected to bridge the clusters. The five gene nodes identified to connect the separate clusters were ptsH, pheA, pfo, CD3092 and CD3640. Next, the 5 bridging nodes and rpoN/sigL/sig54 were added to the 26 gene set and re-entered in the CAT-GXD's ‘Search’ bar. CD3093, a Δsig54 downregulated gene with no initially determined associations, was also added to the network. The addition of CD3092 allowed the connection of CD3093 to the network (FIG. 8). As depicted in FIG. 8, CAT-GxD's network analysis features allows the 28 downregulated genes to be viewed as a network defined by interactions from the STRING database. Two genes without any interactions within the 28 inputted genes, CD1413 and CD3093, are not included in the network. In order to link the separate networks, the 26 genes were sent to the STRING database by clicking on the STRING icon.

### Use Case: STRING Analysis

The STRING database website will pop up in a new window. STRING has the option to add more nodes to the inputted genes by pressing the ‘More” button. Nodes were added a total of 7 times in order to connect 5 of the 6 networks. However, in this example, the 3 gene sporulation membrane/peptidases (CD2697 . . . 1 . . . CD2699 [3117803 . . . 3118351]) cluster was unable to connect to the main network after more than 10 cycles of adding nodes. The least number of nodes with the higher network association scores were selected to bridge the clusters. The 5 nodes were identified to connect the separate clusters are ptsH, pheA, pfo, CD3092 and CD3640. The 5 bridging nodes and rpoN/sigL/sig54 were added to the 26 and re-entered in CAT-GXD's ‘Search’ bar. Also added was CD3093, a Δsig54 downregulated gene with no initially determined associations. Fortunately, the addition of CD3092 was able to connect CD3092 to the network.

One of the more important features of CAT-GxD's network analysis is overlaying gene expression data on the network. Overlaying Δsig54 data, shows the level of downregulation in the 27 genes. No change in gene expression was observed in the 5 bridging nodes. The sigL/sig54/rpoN node appears unchanged in the network but is actually not present in the Δsig54 dataset. The log2 ratio returns an error because of the numerator of the Δsig54/WT ratio being equal to 0. It can be assumed that the deletion of sigL/sig54/rpoN results in downregulation of sigL/sig54/rpoN via its non-expression.

Overlaying the expression ratios on this updated STRING network was highly informative. For the Δsig54/WT ratios, there is downregulation of the 27 genes, but no change in expression for the 5 bridging nodes. The SigL/Sig54/RpoN node is only indicated as a nominal anchor since this gene/protein is absent in the Δsig54 mutant.

With 240 μM DCA treatment of C. difficile for 48 hours, a decrease in Sig54 is readily apparent, and the downregulated expression of 19 of 27 genes (clusters [1] and

) mirrored the pattern observed with the Δsig54 strain. This supports the hypothesis that Sig54 mediates the DCA-induced downregulation of these 19 genes. Unlike in the Δsig54 dataset, however, only 3 of 10 genes of cluster [2] were downregulated. Further, CA exposure results in the downregulation of 4 of the 5 bridging nodes. The fifth and central bridging note, pheA, was upregulated.

Exposing C. difficile to 240 μM DCA for 48 hours produces the most similar expression pattern with Δsig54. 19 of the 27 Δsig54 downregulated genes are also downregulated. Unlike in the Δsig54 dataset, DCA exposure results in the downregulation of 4 of the 5 bridging nodes. The fifth and central bridging note, pheA, was upregulated. SigL/Sig54/RpoN was also observed to be downregulated. It can be hypothesized that DCA exposure results in the downregulation of SigL/Sig54/RpoN which in turn downregulates the CD3093 gene clusters [1] and [3]. Only 3 of 10 genes of cluster [2] was observed to be downregulated

C. difficile treatment with 80 mM succinate for 24 hours results in SigL upregulation, and also the most dissimilar expression pattern relative to Δsig54, with 13 of 27 genes (clusters [1] and [3]) displaying contrasting expression patterns. This supports the hypothesis that Sig54 mediates the succinate-induced upregulation of these 13 genes. Interestingly, 7 of the 10 genes showed similar downregulation patterns with succinate treatment and with Sig54 loss.

Exposing C. difficile to 80 mM succinate for 24 hours produces the most dissimilar expression pattern with Δsig54. Similar to DCA, the central bridging node, pheA, was upregulated and the bridging node of cluster [2], ptsH, was downregulated.

The bridging node of clusters [1] and [3], pfo, and SigL/Sig54/RpoN was upregulated. It can be hypothesized that succinate exposure results to the upregulation of SigL/Sig54/RpoN which in turn upregulates gene clusters [1] and [3]. The effect of DCA and succinate appeared similar in gene cluster [2], where the bridging node, ptsH, is downregulated resulting in partial downregulation of the genes in cluster [2]. The similarity of DCA and succinate gene expression of cluster [2] despite the diverging SigL/Sig54/RpoN levels may indicate that SigL/Sig54/RpoN is not directly involved in this cluster. Similar to DCA, the central bridging node, pheA, was upregulated and the bridging node of cluster [2], ptsH, was downregulated. The bridging node, pfo, of clusters [1] and [3] was upregulated. The similarity of cluster [2] gene expression between DCA and succinate treatment, respectively, despite the diverging Sig54 levels suggests epistatic effects of the two treatments on Sig54-dependent regulation of this group of genes.

### Use Case: KEGG Pathway Analysis

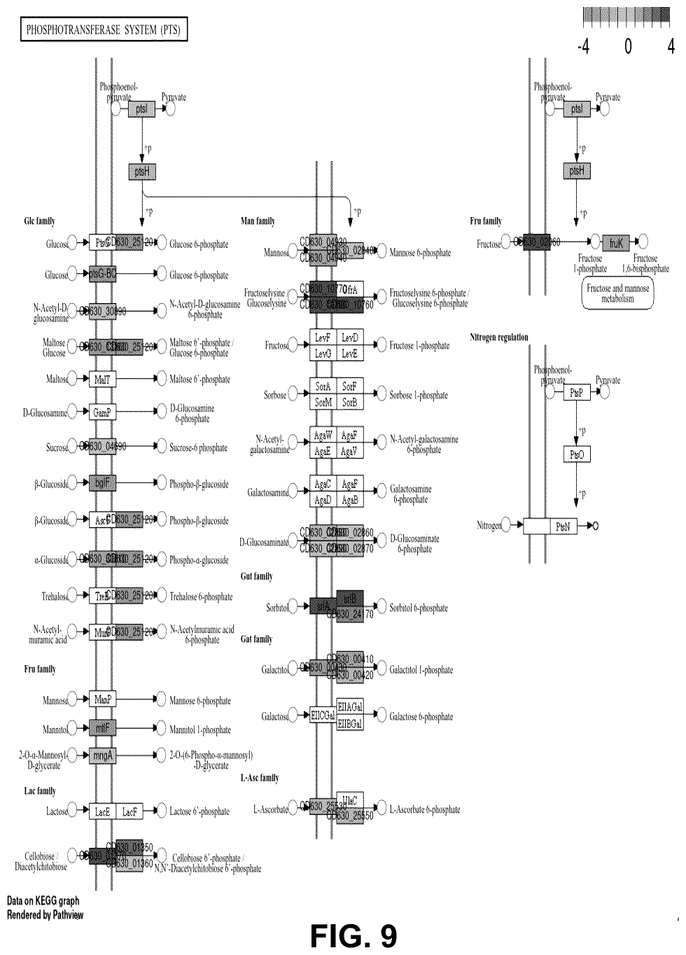

In the pathway tab in CAT-GxD, gene expression data for specific conditions can be overlaid on selected KEGG pathways. Five of the seven PTS genes in cluster [2] are included in KEGG's Phosphotransferase System (PTS) pathway. Four genes are involved in glucosaminate phosphorylation while CD0284 is involved in mannose phosphorylation. The PTS pathway is annotated with colored boxes highlighting sugar phosphorylation processes that distinguish the 3 ratios. Most of the Glc family phosphorylation and L-ascorbate phosphorylation is downregulated in both DCA and succinate, unlike in the Asig54 where it is unchanged. DCA differs from succinate exposure in increased mannitol (mtlF) and decreased sorbitol phosphorylation. Succinate exposure differs from DCA exposure in increased cellobiose, fructoselysine (a fructooligosaccharide) and sorbitol phosphorylation (FIG. 9).

CAT-GxD's pathway analysis gives the user the capability of overlaying gene expression ratios on the selected KEGG pathway. This feature can be used to further investigate the differences in the phosphotransferase system (PTS) gene expression of cluster [2]. 5 of the 7 PTS genes in cluster [2] are included in KEGG's Phosphotransferase System (PTS) pathway. 4 genes are involved in glucosaminate phosphorylation while CD0284 is involved in mannose phosphorylation. The PTS pathway is annotated with colored boxes highlighting sugar phosphorylation processes that distinguish the 3 ratios. They can be used to differentiate DCA or bile acid and succinate induced biofilm formation. Most of the Glc family phosphorylation and L-ascorbate phosphorylation is downregulated in both DCA and succinate, unlike in the Δsig54 where it is unchanged. Where DCA does differ from the succinate is in increased mannitol (mtlF) and decreased sorbitol phosphorylation. Succinate exposure differs from DCA exposure in increased cellobiose, fructoselysine (a fructooligosaccharide) and sorbitol phosphorylation.

These differences may explain the mechanism of why succinate induces C. difficile to produce a significantly thicker biofilm. There are studies have linked increase cellobiose, fructooligosaccharides and sorbitol metabolism and phosphorylation to succinate induced biofilm formation. The diverging regulation of mtIF, a gene involved in mannitol metabolism and phosphorylation, may be a testable difference between DCA and succinate induced biofilm formation. The downregulation of mtlF has been observed in succinate induced biofilms, but its levels have not been identified in DCA or bile acid induced biofilms.

Both DCA and succinate induce biofilm formation in C. difficile with the latter inducing a significantly thicker biofilm. The observed pathway differences may underlie succinate's ability to induce C. difficile to produce significantly thicker biofilms. Previous studies have linked increased cellobiose, fructooligosaccharides and sorbitol metabolism and phosphorylation to succinate induced biofilm formation. The diverging regulation of mtlF, a gene involved in mannitol metabolism and phosphorylation, may be a testable difference between DCA and succinate-induced biofilm formation. The downregulation of mtlF has been observed in succinate induced biofilms but its levels have not been identified in DCA or bile acid induced biofilms.Conclusions

The scope and possibilities of informatics-driven bio-solutions are still meager compared to the scale of the global infectious disease burden. The lack of targeted (e.g., microbiota-sparing) anti-infective strategies over the past decade represents a huge unmet medical need with significant value proposition. CAT-GxD is a powerful platform facilitating easy and customizable analysis of otherwise difficult-to-compare bacterial pathogen gene expression datasets.

The CAT-GxD platform is adaptable for other bacteria as well as bacterium/host response datasets. Ongoing efforts include expansion of CAT-GxD to other CDC Urgent

Threat pathogens ceftriaxone-resistant Neisseria gonorrhoeae, Candida auris, and the two carbapenem-resistant bacterial groups enterobacteriales and Acinetobacter, and CDC Serious Threat bacterial pathogens (diarrheagenic campylobacters and E. coli). While the initial focus is on gene expression, the methodologies encompass broader workflow and goals, including a platform to categorize human host-cell alterations stimulated by enteric pathogens and their virulence factors, e.g., using un-mined datasets of host cell (various cultured cell lines, enteroids, infected mouse tissues, infected human tissues, etc.) as well as RNA and protein changes following treatment/infection with various pathogens.

### REFERENCES

1. Edgar R, Domrachev M, Lash A E. Gene Expression Omnibus: NCBI gene expression and hybridization array data repository. Nucleic Acids Res. 2002;30 (1): 207-10.

2. Barrett T, Wilhite S E, Ledoux P, Evangelista C, Kim I F, Tomashevsky M, et al. NCBI GEO: Archive for functional genomics data sets—update. Nucleic Acids Res. 2012;41 (D1): D991-D5.

3. Vizcaíno J A, Deutsch E W, Wang R, Csordas A, Reisinger F, Ríos D, et al. ProteomeXchange provides globally coordinated proteomics data submission and dissemination. Nat Biotechnol. 2014;32 (3): 223-6.

4. Deutsch E W, Bandeira N, Sharma V, Perez-Riverol Y, Carver J J, Kundu DJ, et al. The ProteomeXchange consortium in 2020: Enabling ‘big data’ approaches in proteomics. Nucleic Acids Res. 2019;48 (D1): D1145-D52.

5. Deutsch E W, Csordas A, Sun Z, Jarnuczak A, Perez-Riverol Y, Ternent T, et al. The ProteomeXchange consortium in 2017: Supporting the cultural change in proteomics public data deposition. Nucleic Acids Res. 2016;45 (D1): D1100-D6.

6. Wilkinson M D, Dumontier M, Aalbersberg I J, Appleton G, Axton M, Baak A, et al. The FAIR guiding principles for scientific data management and stewardship. Sci Data. 2016;3:160018.

7. Finn E, Andersson F L, Madin-Warburton M. Burden of Clostridioides difficile infection (CDI)—a systematic review of the epidemiology of primary and recurrent CDI. BMC Infect Dis. 2021;21 (1): 456.

8. Bartlett J G. Clinical practice: Antibiotic-associated diarrhea. N Engl J Med. 2002;346 (5): 334-9.

9. Zhang S, Palazuelos-Munoz S, Balsells E M, Nair H, Chit A, Kyaw M H. Cost of hospital management of Clostridium difficile infection in United States—a meta—analysis and modelling study. BMC Infect Dis. 2016; 16 (1): 447.

10. Feuerstadt P, Theriault N, Tillotson G. The burden of CDI in the United States: A multifactorial challenge. BMC Infect Dis. 2023;23 (1): 132.

11. Ojemolon PE, Shaka H, Kwei-Nsoro R, Laswi H, Ebhohon E, Shaka A, et al. Trends and disparities in outcomes of Clostridioides difficile infection hospitalizations in the United States: A ten-year joinpoint trend analysis. J Clin Med Res. 2022;14 (11): 474-86.

12. Olson R D, Assaf R, Brettin T, Conrad N, Cucinell C, Davis J J, et al. Introducing the Bacterial and Viral Bioinformatics Resource Center (BV-BRC): a resource combining PATRIC, IRD and ViPR. Nucleic Acids Res. 2023;51 (D1): D678-d89.

13. Arrieta-Ortiz M L, Immanuel S R C, Turkarslan S, Wu W-J, Girinathan B P, Worley J N, et al. Predictive regulatory and metabolic network models for systems analysis of Clostridioides difficile. Cell Host Microbe. 2021;29 (11): 1709-23.e5.

14. Sebaihia M, Wren B W, Mullany P, Fairweather N F, Minton N, Stabler R, et al. The multidrug-resistant human pathogen Clostridium difficile has a highly mobile, mosaic genome. Nat Genet. 2006;38 (7): 779-86.

15. Wüst J, Sullivan N M, Hardegger U, Wilkins T D. Investigation of an outbreak of antibiotic-associated colitis by various typing methods. J Clin Microbiol. 1982;16 (6): 1096-101.

16. Riedel T, Bunk B, Thürmer A, Spröer C, Brzuszkiewicz E, Abt B, et al. Genome resequencing of the virulent and multidrug-resistant reference strain Clostridium difficile 630. Genome Announc. 2015;3 (2): 10.1128/genomea. 00276-15.

17. Monot M, Boursaux-Eude C, Thibonnier M, Vallenet D, Moszer I, Medigue C, et al. Reannotation of the genome sequence of Clostridium difficile strain 630. J Med Microbiol. 2011;60 (8): 1193-9.

18. Chang W C J, Allaire J, Sievert C, Schloerke B, Xie Y, Allen J, McPherson J, Dipert A, Borges B. shiny: Web application framework for R. 2023.

19. Wickham H F R, Henry L, Müller K, Vaughan D. dplyr: A Grammar of Data Manipulation. R package version 1.1.4. ed2023.

20. Luna A, Shah O, Sander C, Shannon P. cyjShiny: A cytoscape.js R Shiny Widget for network visualization and analysis. PLOS One. 2023;18 (8): e0285339.

21. Galili T, O'Callaghan A, Sidi J, Sievert C. heatmaply: An R package for creating interactive cluster heatmaps for online publishing. Bioinform. 2017;34 (9): 1600-2.

22. Wickam H. ggplot2: Elegant graphics for data analysis. Version 3.4.4. ed: Springer-Verlag New York; 2016.

23. Luo W, Brouwer C. Pathview: an R/Bioconductor package for pathway-based data integration and visualization. Bioinform. 2013;29 (14): 1830-1.

24. Tenenbaum D M B. KEGGREST: Client-side REST access to the Kyoto Encyclopedia of Genes and Genomes (KEGG). R package version 1.42.0. ed2023.

25. Müller K W H, James D A, Falcon S. RSQLite: SQLite interface for R. Version 2.3.4. ed2023.

26. Cunningham F, Allen J E, Allen J, Alvarez-Jarreta J, Amode M R, Armean Irina M, et al. Ensembl 2022. Nucleic Acids Res. 2021;50 (D1): D988-D95.

27. Sayers E W, Bolton E E, Brister J R, Canese K, Chan J, Comeau D C, et al. Database resources of the national center for biotechnology information. Nucleic Acids Res. 2022;50 (D1): D20-d6.

28. Yu N Y, Wagner J R, Laird M R, Melli G, Rey S, Lo R, et al. PSORTb 3.0: Improved protein subcellular localization prediction with refined localization subcategories and predictive capabilities for all prokaryotes. Bioinform. 2010;26 (13): 1608-15.

29. Norsigian C J, Danhof H A, Brand C K, Oezguen N, Midani F S, Palsson B O, et al. Systems biology analysis of the Clostridioides difficile core-genome contextualizes microenvironmental evolutionary pressures leading to genotypic and phenotypic divergence. npj Syst Biol Appl. 2020;6 (1): 31.

30. Galperin M Y, Makarova K S, Wolf Y I, Koonin E V. Expanded microbial genome coverage and improved protein family annotation in the COG database. Nucleic Acids Res. 2015;43 (Database issue): D261-9.

31. Tatusov R L, Koonin E V, Lipman D J. A genomic perspective on protein families. Science. 1997;278 (5338): 631-7.

32. Kanehisa M. Toward understanding the origin and evolution of cellular organisms. Protein Sci. 2019;28 (11): 1947-51.

33. Kanehisa M, Furumichi M, Sato Y, Kawashima M, Ishiguro-Watanabe M. KEGG for taxonomy-based analysis of pathways and genomes. Nucleic Acids Res. 2023;51 (D1): D587-d92.

34. Kanehisa M, Goto S. KEGG: Kyoto Encyclopedia of Genes and Genomes. Nucleic Acids Res. 2000;28 (1): 27-30.

35. Aziz R K, Bartels D, Best A A, DeJongh M, Disz T, Edwards R A, et al. The RAST Server: Rapid Annotations using Subsystems Technology. BMC Genom. 2008;9 (1): 75.

36. Brettin T, Davis J J, Disz T, Edwards R A, Gerdes S, Olsen G J, et al. RASTtk: A modular and extensible implementation of the RAST algorithm for building custom annotation pipelines and annotating batches of genomes. Sci Rep. 2015;5 (1): 8365.

37. Overbeek R, Olson R, Pusch G D, Olsen G J, Davis J J, Disz T, et al. The SEED and the Rapid Annotation of microbial genomes using Subsystems Technology (RAST). Nucleic Acids Res. 2014;42 (Database issue): D206-14.

38. Consortium T U. UniProt: The universal protein knowledgebase in 2023. Nucleic Acids Res. 2022;51 (D1): D523-D31.

39. Aleksander S A, Balhoff J, Carbon S, Cherry J M, Drabkin H J, Ebert D, et al. The Gene Ontology knowledgebase in 2023. Genetics. 2023;224 (1).

40. Ashburner M, Ball C A, Blake J A, Botstein D, Butler H, Cherry J M, et al. Gene ontology: Tool for the unification of biology. The Gene Ontology Consortium. Nat Genet. 2000;25 (1): 25-9.

41. Szklarczyk D, Kirsch R, Koutrouli M, Nastou K, Mehryary F, Hachilif R, et al. The STRING database in 2023: Protein-protein association networks and functional enrichment analyses for any sequenced genome of interest. Nucleic Acids Res. 2023;51 (D1): D638-d46.

42. Jumper J, Evans R, Pritzel A, Green T, Figurnov M, Ronneberger O, et al.

Highly accurate protein structure prediction with AlphaFold. Nature. 2021;596 (7873): 583-9.

43. Rangwala S H, Kuznetsov A, Ananiev V, Asztalos A, Borodin E, Evgeniev V, et al. Accessing NCBI data using the NCBI Sequence Viewer and Genome Data Viewer (GDV). Genome Res. 2021;31 (1): 159-69.

It is understood that the disclosed method and compositions are not limited to the particular methodology, protocols, and reagents described as these can vary. It is also to be understood that the terminology used herein is for the purpose of describing particular forms only, and is not intended to limit the scope of the present invention which will be limited only by the appended claims.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a nucleic acid sequence is disclosed and discussed and a number of modifications that can be made to a number of molecules including the nucleic acid sequence are discussed, each and every combination and permutation of the nucleic acid sequence and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

It must be noted that as used herein and in the appended claims, the singular forms “a”, “an”, and “the” include plural reference unless the context clearly dictates otherwise. Thus, for example, reference to “a nucleic acid sequence” includes a plurality of such nucleic acids, reference to “the nucleic acids” is a reference to one or more nucleic acid and equivalents thereof known to those skilled in the art, and so forth. “Optional” or “optionally” means that the subsequently described event, circumstance, or material may or may not occur or be present, and that the description includes instances where the event, circumstance, or material occurs or is present and instances where it does not occur or is not present.

Unless the context clearly indicates otherwise, use of the word “can” indicates an option or capability of the object or condition referred to. Generally, use of “can” in this way is meant to positively state the option or capability while also leaving open that the option or capability could be absent in other forms or forms of the object or condition referred to. Unless the context clearly indicates otherwise, use of the word “may” indicate an option or capability of the object or condition referred to. Generally, use of “may” in this way is meant to positively state the option or capability while also leaving open that the option or capability could be absent in other forms or forms of the object or condition referred to. Unless the context clearly indicates otherwise, use of “may” herein does not refer to an unknown or doubtful feature of an object or condition.

Ranges can be expressed herein as from “about” one particular value, and/or to “about” another particular value. When such a range is expressed, also specifically contemplated, and considered disclosed is the range from the one particular value and/or to the other particular value unless the context specifically indicates otherwise. Similarly, when values are expressed as approximations, by use of the antecedent “about,” it will be understood that the value forms another, specifically contemplated form that should be considered disclosed unless the context specifically indicates otherwise. It will be further understood that the endpoints of each of the ranges are significant both in relation to the other endpoint, and independently of the other endpoint unless the context specifically indicates otherwise. All of the individual values and sub-ranges of values contained within an explicitly disclosed range are also specifically contemplated and should be considered disclosed unless the context specifically indicates otherwise. Finally, all ranges refer both to the recited range as a range and as a collection of individual numbers from and including the first endpoint to and including the second endpoint. In the latter case, any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these forms are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. can include numerous options and alternatives, this should not be construed as, and is not an admission that, such options and alternatives are equivalent to each other or, in particular, are obvious alternatives. Thus, for example, a list of different gene targets does not indicate that the listed gene targets are obvious one to the other, nor is it an admission of equivalence or obviousness.

Every component disclosed herein is intended to be and should be considered to be specifically disclosed herein. Further, every subgroup that can be identified within this disclosure is intended to be and should be considered to be specifically disclosed herein. As a result, it is specifically contemplated that any component, or subgroup of components can be either specifically included for or excluded from use or included in or excluded from a list of components.

Those skilled in the art will recognize, or be able to ascertain using no more than routine experimentation, many equivalents to the specific forms of the method and compositions described herein. Such equivalents are intended to be encompassed by the following claims.

We claim:

1. A method for analysis of the homology and/or differential expression of one or more user-defined genes and/or gene functions from a pool of genomic sequence data derived from a multiplicity of samples of organisms of the same species, or for a host-pathogen interaction, the method comprising
(i) determining homology between the multiplicity of samples to identify common genes;
(ii) identifying differential expression of the common genes; and
(iii) presenting data reporting the homology and/or differential expression of the user-defined genes and/or gene functions,
wherein the method is implemented on a computer, and
wherein the pool of genomic sequence data is provided in the form of a computer-readable database(s).

2. The method of claim 1, wherein the presenting in step (iii) comprises identifying relationships between one or more genes within samples of the multiplicity of samples.

3. The method of claim 1, wherein the computer-readable database comprises one or more of gene expression data, transcriptomics data, protein abundance data, and proteomics data.

4. The method of claim 3, wherein the database comprises one or more selected from the group consisting of the Gene Expression Omnibus (GEO) database, the ProteomeXchange database, the Gene Ontology (GO) database, KEGG (Krypto Encyclopedia of Genes and Genomics) database, the BioCyc Genome Database Collection, Database for Annotation, Visualization and Integrated Discovery (DAVID) bioinformatics database, RAST Subsystems database (Clusters of Orthologous Genes), UniProt, and Enzyme Classes.

5. The method of claim 3, wherein steps (i) and/or (ii) comprise one or more of:
(a) determining sequence homology between two or more of the multiplicity of samples of the organism;
(b) determining differential gene expression and/or abundance, comprising log2 ratio, median normalization and one-sample t-test between two or more of the multiplicity of samples of the organism;
(c) principal component analysis (PCA); and
(d) data structuring, comprising column identity and position for data points.

6. The method of claim 3, wherein steps (i) and/or (ii) comprise the creating, storing, updating and/or retrieving of data comprising quantitative proteomic analysis and/or transcriptomic analysis of two or more samples as a relational database using structured query language (SQL).

7. The method of claim 1, wherein presenting data in step (iii) is implemented through one or more computer programs comprising RShiny, RStudio Connect, gene set enrichment analysis (GSEA).

8. The method of claim 7, wherein presenting data comprises providing a heat map representation of changes in the expression of one or more genes amongst two or more of the multiplicity of samples.

9. The method of claim 1, wherein the analysis is initiated by one or more user-defined input parameters entered through a user interface connected with the computer.

10. The method of claim 9, wherein one or more user-defined input parameter is selected from the group consisting of the name of a gene, a gene function, a gene mutation, a nucleic acid sequence, the name of an organelle, a genetic pathway, a sub-species or clade, the name of a polypeptide, an amino acid sequence, a gene expression pathway, the name of a toxin, the name of a disease or disorder, the name of a virus, the name of a geographic location or place, a date, a range of dates, the name of a drug, and a host cell or organism, the name of an investigator or scientific institution, a type/classification of a study, a gene expression log2 ratio, a keyword search term, an enzyme class, gene grouping, and the name of a methodology; or combinations thereof.

11. The method of claim 10, wherein the one or more user-defined input parameter comprises the name of a gene, or a code corresponding to a gene, and
(a) the presenting in step (iii) comprises displaying one or more polymorphisms within the gene in the pool of genomic sequence data,
optionally wherein the presenting also displays one or more sample(s) associated with each polymorphism; or
(b) the presenting in step (iii) comprises displaying differential expression of the gene amongst the pool of genomic sequence data.

12. The method of claim 9, wherein the one or more user-defined input parameter comprises
(a) a genomic subset selected from the group consisting of pangenome, core genome, metabolic core genome, and essential genes; and/or
(b) a cellular location selected from the group consisting of the cell cytoplasm and the cytoplasmic membrane;
(c) an experiment parameter filter selected from the group consisting of organism-specific genes, response to specific gene knockout, bile acid, antibacterial, antibiotic, and stress; and/or
(d) a search term from a database selected from the group consisting of KEGG (Krypto Encyclopedia of Genes and Genomics), GO (Gene Ontology), COG (Clusters of Orthologous Genes), the BioCyc Genome Database Collection, Database for Annotation, Visualization and Integrated Discovery (DAVID) bioinformatics database, RAST (Rapid Annotations using Subsystems Technology) and UNIPROTKB.

13. The method of claim 1, wherein the organism is a microorganism selected from the group consisting of a bacterium, a virus, a fungi, a protozoan, an algae and an archaebacterium,
optionally wherein the microorganism is a pathogenic microorganism associated with one or more diseases or disorders in humans,
optionally wherein the pathogenic microorganism is Clostridioides difficile or Neisseria gonorrhoeae.

14. The method of claim 5, further comprising
(iv) determining or correlating gene expression of one or more genes expressed by the organism that is known to be associated with resistance or susceptibility to one or more active agents,
wherein an increase in gene expression compared to a reference or median gene expression selects the organism as having an increased chance of survival in the presence of the one or more active agents, and/or
wherein an reduction in gene expression or lack of change of gene expression compared to a reference or median gene expression selects the organism as having an reduced chance of survival in the presence of the one or more active agents.

15. The method of claim 14, wherein
(a) the gene expression value is determined by calculating a log2 gene expression value,
optionally wherein a positive log2 fold change corresponds to increased expression, whereas negative values correspond to decreased expression; and/or
(b) the median or reference value is determined by expression of genes of a reference or control dataset.

16. The method of claim 15, further comprising
(v) correlating treatment options for an infection of a subject with the organism(s) with changes in gene expression to inform a likelihood of positive clinical outcome in the subject when treated with one or more therapeutic agents that are associated with the one or more genes expressed by the organism.

17. The method of claim 16, wherein an increase in gene expression compared to a reference or median gene expression indicates that the subject has a lower likelihood of a positive clinical outcome when treated with one or more therapeutic agents that are associated with the one or more genes expressed by the organism.

18. The method of claim 17, wherein the positive clinical outcome comprises therapeutic efficacy of the therapeutic agent and/or survival of the subject.

19. The method of claim 16, further comprising
(vi) treating a subject in need thereof for an infection with the organism by administering to the subject the therapeutic agent in an amount effective to treat the infection if the organism has a negative log2 fold change.

20. A method for identifying molecular pathways in one or more pathogenic microbial strains, wherein the pathogenic microbial strain is from a species selected from the group consisting of Clostridioides spp., Neisseria spp., Candida spp., Enterobacteriaceae spp., Acinetobacter spp., Campylobacter spp., and Escherichia spp. in response to an active agent, comprising
(a) contacting a first microbial strain of the pathogenic microbe species with a first active agent;
(b) determining a change in gene expression for one or more genes of the first microbial strain in the presence of the active agent,
wherein the determining optionally further comprises evaluating gene expression and dose response data for the first active agent,
optionally wherein the first active agent is an antimicrobial agent, and
wherein the phenotypic and/or genotypic responses to the first active agent comprise susceptibility or resistance to the antimicrobial agent;
(c) analyzing genes that demonstrate expression and dose response correlations to identify significantly represented molecular pathways,
wherein a change in gene expression greater or lower than a reference or median value identifies genes in the first microbial strain responsive to the first active agent,
optionally wherein the control value is the gene expression of a wild type strain of the first microbial strain in the absence of the active agent;
(d) determining differences in gene expression between the first microbial strain and a second or further microbial strain of the same species in the presence of the same active agent,
wherein the differences identify molecular pathways that are responsible for different phenotypic and/or genotypic responses to the first active agent;
(e) compiling the gene expression data for the first and second or further microbial strain in the presence of the first active agent in a database,
wherein the data base is searchable,
optionally wherein the database further comprises gene expression data for a multiplicity of different microbial strains, and/or a multiplicity of different active agents; and
(f) Optionally treating a subject having an infection caused the first microbial strain with the first active agent when the pathogenic microorganism comprises molecular pathways that are associated with susceptibility to the first active agent,
wherein the molecular pathways of the pathogenic microorganism are determined by comparing gene expression data of the pathogenic microorganism to those in the database; or
not treating a subject with the first active agent when the subject has an infection caused by a pathogenic microorganism comprising molecular pathways that are responsible for resistance to the first antimicrobial agent,
wherein the molecular pathways of the pathogenic microorganism are determined by comparing gene expression data of the pathogenic microorganism to those in the database.
